(19) United States
(12) Patent Application Publication
(10) Pub. No.: US 2023/0203523 A1
(43) Pub. Date: Jun. 29, 2023

(54) HPPD INHIBITOR HERBICIDE TOLERANT PLANT

(71) Applicant: Advanta Holdings B.V., Breda (NL)

(72) Inventors: Pedro Alejandro Pardo, Rosalia de Lerma (AR); Hernan Gabriel Bondino, College Station, TX (US); Maria de la Paz Arrieta Montiel, College Station, TX (US)

(21) Appl. No.: 18/088,899

(22) Filed: Dec. 27, 2022

Related U.S. Application Data
(60) Provisional application No. 63/294,114, filed on Dec. 28, 2021.

(51) Int. Cl. C12N 15/82; C07K 14/415

(52) U.S. Cl. CPC C12N 15/8274; C07K 14/415

(57) ABSTRACT

Described herein is an amino acid sequence having SEQ ID NO: 3, including a mutation from a Tryptophane to a Serine residue at position 445. Also described are nucleic acids encoding the amino acid sequence, and plants or seeds including the amino acid sequence such as a sorghum plant or seed. In an aspect, the plant or seed has enhanced resistance or tolerance to an HPPD herbicide.

### CROSS-REFERENCE TO RELATED APPLICATIONS

This application claims priority to U.S. Provisional Application 63/294,114 filed on Jan. 28, 2021, which is incorporated herein by reference in its entirety.

### SEQUENCE LISTING

The Instant Application contains a Sequence Listing which has been submitted electronically in XML format and is hereby incorporated by reference in its entirety. Said XML copy, created on Dec. 23, 2022, is named “AOD0018US2” and is 44,683 bytes in size.

### FIELD OF INVENTION

The present invention relates to a novel mutation conferring HPPD herbicide tolerance or resistance in a plant. The present invention also relates to a plant comprising a mutation which confers HPPD herbicide tolerance or resistance. The invention also relates to a method of producing such a plant, and a method of identifying and selecting such a plant. The invention also relates to progeny, plant parts, plant tissues and plant seeds of such a plant. The invention also provides related methods of using the plants, parts thereof, and the mutations described herein.

### BACKGROUND OF INVENTION

The 4-hydroxyphenylpyruvate dioxygenases (HPPDs) are enzymes which catalyze the reaction in which para-hydroxyphenylpyruvate (abbreviated herein as HPP), a tyrosine degradation product, is transformed into homogentisate (abbreviated herein as HG), the precursor in plants of tocopherol and plastoquinone (Crouch N. P. et al. (1997), Tetrahedron, 53, 20, 6993-7010, Fritze et al. (2004), Plant Physiology 134:1388-1400). Tocopherol acts as a membrane-associated antioxidant. Plastoquinone, firstly acts as an electron carrier between PSII and the cytochrome b6/f complex and secondly, is a redox cofactor for phytoene desaturase, which is involved in the biosynthesis of carotenoids.

While the overall products of this cycle are used to create energy, plants and higher order eukaryotes utilize HPPD for a much more important reason. In eukaryotes, HPPD is used to regulate blood tyrosine levels, and plants utilize this enzyme to help produce the cofactors plastoquinone and tocopherol which are essential for the plant to survive (Introduction to Plant Biochemistry, 1998).

When HPPD is inhibited, it leads to photosynthesis uncoupling, accessory light-harvesting pigments deficiency and, destruction of chlorophyll by UV-radiation and reactive oxygen species (bleaching) due to the lack of photo protection normally provided by carotenoids, thus leading to plant death.

There are some molecules that inhibit HPPD and are known as HPPD inhibiting herbicides. There are various HPPD inhibiting herbicides that are commercially available and are important to control unwanted weeds in the field. To extend the scope of use of these herbicides there is a consistent effort to develop commercially important plant varieties that are resistant to such herbicides without underperforming other metabolic tolerance.

Patent application WO 2009/144079 discloses a mutated hydroxyphenylpyruvate dioxygenase (HPPD) at position 336 of the Pseudomonas fluorescens HPPD protein and its use for obtaining plants which are tolerant to HPPD inhibitor herbicides.

Patent application WO 2008/150473 discloses two distinct tolerance mechanisms—a modified Avena sativa gene coding for a mutant HPPD enzyme and a CYP450 Maize monooxygenase (nsf1 gene)—was exemplified to obtain an improved tolerance to HPPD inhibitor herbicides, but no data have been disclosed demonstrating the synergistic effects based on the combination of both proteins.

U.S. Pat. No. 8,853,496B2 discloses sequences encoding a hydroxyphenylpyruvate dioxygenase (EC 1.13.11.27, abbreviated herein as HPPD) obtained from Blepharisma japonicum and the use of such nucleic acid sequences, proteins or chimeric genes for obtaining plants which are tolerant to HPPD inhibitor herbicides.

Despite all successful efforts of developing plants that are resistant or tolerant to HPPD inhibitor herbicides, it is still needed to develop or to improve the tolerance of plants to the available HPPD herbicides.

### SUMMARY OF THE INVENTION

The present invention relates to a novel mutation which has been found to impart resistance or tolerance to an HPPD herbicide in a plant. The novel mutation encodes a serine residue at amino acid position 445 of SEQ ID NO: 3. The presence of the serine residue at position 445 in SEQ ID NO: 3 has been shown to provide resistance or tolerance to an HPPD herbicide in a plant. In an embodiment, the novel mutation is a point mutation at position 445 of the amino acid sequence of SEQ ID NO: 3. In an embodiment, the novel mutation is a substitution mutation at position 445 of the amino acid sequence of SEQ ID NO: 3, where tryptophan is replaced with serine. The novel mutation as defined herein may be referred to herein as the mutation of the invention or the ADV-HT2 mutation. The novel mutation may be present in a non-HPPD gene located on chromosome 3. The novel mutation may be encoded by a nucleic acid sequence present in a region of chromosome 3, which encodes the amino acid sequence of SEQ ID NO: 3 comprising a serine residue at position 445. In an embodiment, a nucleic acid sequence which encodes the amino acid sequence of SEQ ID NO: 3 comprising a serine residue at position 445 may be the sequence of SEQ ID NO: 1 or may comprise a sequence which encodes a sequence substantially identical thereto and having a serine residue at a position corresponding to amino acid position 445 of SEQ ID NO: 3. Such a substantially identical sequence may have one of Alternative Codons 1-5 at the allele coding for the serine residue at position 445.

The invention also relates to a genetic marker that is indicative of a phenotype associated with HPPD herbicide tolerance or resistance. The genetic marker may be the mutation of the invention, or a marker which is in linkage disequilibrium with the mutation of the invention. A mutation which is in linkage disequilibrium with an ADV-HT2 mutation of the invention is the presence of the “C” at position 51 of SEQ ID NO: 8, which is indicative of HPPD-type herbicide resistance.

The herbicide-resistant or tolerant plants of the invention comprise a serine at position 445 in an amino acid sequence encoded by a nucleic acid sequence on chromosome 3. The nucleic acid sequence may comprise the nucleic acid sequence of SEQ ID NO: 1 or may be substantially identical to the sequence of SEQ ID NO: 1, and/or may have alternate codons that codes for the novel mutation encodes a serine residue at amino acid position 445 of SEQ ID NO: 3 The polypeptide is a non-HPPD polypeptide. In particular, the plants, plant parts, plant tissues, and plant seeds of the invention have an increased resistance to HPPD herbicides when compared to a wild-type plant. A herbicide-resistant or tolerant plant of the invention may comprise a mutation on chromosome 3, comprising a G to C substitution at position 51 of SEQ ID NO: 8.

The present invention also provides a method of producing a herbicide-resistant or tolerant plant of the invention. A method of producing a herbicide resistant or tolerant plant of the invention may comprise introducing the mutation of the invention into a plant.

The present invention provides a method of producing a plant that comprises resistance or tolerance to HPPD herbicide, wherein the method comprises the cross-pollination of a first plant with a second plant so as to produce hybrid seeds. The seeds can be sown and allowed to grow into a hybrid plant, particularly an F1 hybrid plant. Suitably, both the first and second parent plants comprise in their genome at least one copy of a mutation of SEQ ID NO: 1 or any Alternate Codons which encodes a serine residue at amino acid position 445 of SEQ ID NO: 3. Preferably, both the first and second parent plants are homozygous for a mutation in the codon encodes a serine residue at amino acid position 445 of SEQ ID NO: 3.

The present invention further provides a method of detecting an allele or gene of the novel mutation of the invention in a plant. Such a diagnostic detection method may for example involve polymerase chain reaction (PCR) amplification of specific regions of the plant located on chromosome no. 3 using primers designed to anneal to specific sites within the gene of interest, for example at sites that are at the vicinity of the mutation. The regions which may be detected include a mutant allele of the ADV-HT2 allele as described herein, and/or a mutation on chromosome 3, comprising a G to C substitution at position 51 of SEQ ID NO: 8. Primers and kits for the detection of such mutation are also provided.

The present invention also provides a method of controlling weeds that grow in the vicinity of a plant. Such a plant may comprise a mutation of the invention and is resistance or tolerance to an HPPD herbicide. A weed may include, but is not limited to, Grasses-weeds: Echinochloa colona, Echicochloa crus-galli, Digitaria sanguinalis, Eleusine indica; Broad leaf weeds: Anoda cristata, Ipomoea purpurea, Amaranthus quitensis, Amaranthus hybridus, Portulaca oleracea, Raphanus sativus, Brassica campestris, Chenopodium album, Tagetes minuta, Datura ferox and Parasitic Weed. A method of controlling the weeds that grows in the vicinity of a plant may comprise the application of effective amount of an HPPD herbicide to the weeds and to the plant, which suitably may be a HPPD herbicide resistant or tolerant plant comprising the novel mutation of the invention.

The present invention further provides a method of selection of a tolerant or resistant plant comprising the novel mutation of the invention. Such a method may comprise applying an effective amount of HPPD herbicide on a one or more plants and selecting the plant that survives the application of such herbicide.

The invention also provides one or more QTLs which map to a novel chromosomal region and to genetic markers that are indicative of phenotypes associated with HPPD herbicide tolerance. A genetic marker may be the mutation of the invention, or a marker which is linked to the mutation of the invention. A marker which is linked to a marker of the invention may comprise a mutation on chromosome 3, comprising a G to C substitution at position 51 of SEQ ID NO: 8.

An aspect of the present invention is to provide a novel gene mutation imparting HPPD herbicide tolerance or resistance. The novel gene mutation may be the mutation of the invention, as defined herein.

Another aspect of the present invention is a HPPD herbicide tolerant or resistance plant, plant part, plant tissue and plant seed. A HPPD herbicide tolerant or resistance plant, plant part, plant tissue and plant seed may comprise the mutation of the invention. Preferably the plant, plant part, plant tissue or plant seed is a sorghum plant and part, tissue, or seed thereof.

In another aspect the present invention provides a plant, plant part, plant tissue and plant seed that contain wild type HPPD enzyme and still confers resistance or tolerance to HPPD herbicide. Suitably, a plant, plant part, plant tissue or plant seed of this aspect comprises a mutation of the invention.

Another aspect of the present invention is a HPPD herbicide tolerant plant that comprises a nucleic acid sequence which encodes a protein comprising the sequence of SEQ ID NO: 3. The nucleic acid sequence may be a sequence which is substantially identical to SEQ ID NO: 1 or may comprise a sequence which codes for SEQ ID NO: 3 or a sequence substantially identical thereto and having a serine residue at a position which corresponds to amino acid position 445 of SEQ ID NO: 3.

Yet another aspect of the invention is a nucleic acid sequence comprising a mutation corresponding to the mutation in sorghum line with accession number NCIMB 43919 (deposited under the Budapest Treaty on 10 Dec. 2021) with which confers HPPD herbicide resistance or tolerance.

Another aspect of the invention is one or more QTLs that can be used to identify and detect the novel mutation in chromosome 3 of Sorghum plant that confer HPPD herbicide resistance or tolerance.

Yet another aspect of the invention is a nucleic acid sequence comprising a mutation comprises an alternate codon of SEQ ID NO: 1 or comprises a sequence which codes for SEQ ID NO: 3 or a sequence substantially identical thereto and having a serine residue at a position which corresponds to amino acid position 445 of SEQ ID NO: 3. The mutation may be a mutation of the invention as defined above.

Another aspect of the invention is to provide one or more molecular markers that can be used to identify and detect the novel mutation in chromosome 3 of Sorghum plant that produces HPPD herbicide resistance or tolerance. The novel mutation may be a mutation of the invention as defined above. A suitable marker may be a mutation on chromosome 3, comprising a G to C substitution at position 51 of SEQ ID NO: 8., which is in linkage disequilibrium with the ADV-HT2 allele of the invention.

A suitable marker to detect mutation on chromosome 3, may comprise the alternate codons which is in linkage disequilibrium with the ADV-HT2 allele of the invention.

Another aspect of the invention is to provide method and kits to detect a mutation of the invention or an allele in linkage disequilibrium therewith. A method or kit may detect a mutation of SEQ ID NO: 1 or a sequence substantially identical thereto, wherein the mutation encodes a serine residue at position 445 of SEQ ID NO: 3. The mutation may be an Alternate Codons which encodes a serine residue at position 445 of SEQ ID NO: 3. The mutation may be a mutation of the invention as defined above. A method or kit may detect a mutation on chromosome 3, comprising a G to C substitution at position 51 of SEQ ID NO: 8.

Another aspect of the invention is a method of breeding a novel mutant plant to provide a hybrid germplasm that confers resistance or tolerance to inhibition by one or more HPPD herbicide at levels of said one or more HPPD herbicide would normally inhibit the growth of a hybrid.

Another aspect of the invention is a method of using an HPPD herbicide to kill or reduce the viability of an unwanted plant in a field planted with a plant of the invention.

### BRIEF DESCRIPTION OF THE DRAWINGS

FIG. 1: Represents biomass accumulation for 2 grain sorghum genotypes (BV94-4045, wild type and BV10-11924, mutant ADV-HT2) 20 days after herbicide treatment expressed as Biomass percentage of Control treatment for Control, Topramezone 1×, 2×, and 4× treatments. Error bars represent 2*Standard error.

FIG. 2: Represents biomass accumulation for 2 grain sorghum genotypes (BV94-4045, wild type and BV10-11924, mutant ADV-HT2) 20 days after herbicide treatment expressed as Biomass percentage of Control treatment for Control, Mesotrione 1×, 2×, and 4× treatments. Error bars represent 2*Standard error

FIG. 3: Represents biomass accumulation for 2 grain sorghum genotypes (BV94-4045, wild type and BV10-11924, mutant ADV-HT2) 20 days after herbicide treatment expressed as Biomass percentage of Control treatment for Control, Tolpyralate 1×, 2×, and 4× treatments. Error bars represent 2*Standard error.

FIG. 4: Represents biomass accumulation for 2 grain sorghum genotypes (BV94-4045, wild type and BV10-11924, mutant ADV-HT2) 20 days after herbicide treatment expressed as Biomass percentage of Control treatment for Control, Isoxaflutole 1×, 2×, and 4× treatments. Error bars represent 2*Standard error.

FIG. 5: Represents biomass accumulation for 2 grain sorghum genotypes (BV94-4045, wild type and BV10-11924, mutant ADV-HT2) 20 days after herbicide treatment expressed as Biomass percentage of Control treatment for Control, Byciclopirona 1×, 2×, and 4× treatments. Error bars represent 2*Standard error

FIG. 6: Represents the QTLs region to detect the present mutation.

### DETAILED DESCRIPTION OF THE INVENTION

It is to be understood that this invention is not limited to particular embodiments or examples, which can of course vary. It is also to be understood that the terminology used herein is for the purpose of describing particular embodiments only and is not intended to be limiting.

### Definitions

As used in the specification and the appended claims, terms in the singular and the singular forms “a”, “an” and “the”, for example, include plural referents unless the content clearly dictates otherwise. Thus, for example, reference to “plant”, “the plant” or “a plant” also includes plurality of plants; also, depending on the context, use of the term “plant” can also include genetically similar or identical progeny of that plants; use of the term “a nucleic acid” optionally includes, as a practical matter, many copies of that nucleic acid molecule; similarly, the term “probe” optionally and typically encompasses many similar or identical probe molecules.

Certain definitions used in claims and description are defined below to provide a clear and consistent understanding of the specification and claims including the scope to be given such terms:

Allele: refers to an allele in any of one or more alternative forms of a gene which relate to one trait or characteristic. In diploid cell or organism, the two alleles of a given gene occupy corresponding loci on a pair of homologous chromosomes. With regard to SNP marker, allele refers to the specific nucleotide base present at the SNP locus in that individual plant.

Germplasm: Germplasm means the genetic material that comprises the physical foundation of the hereditary qualities of an organism. Germplasm includes seeds and living tissue from which new plants may be grown; or, another plant part, such a leaf, stem, pollen, or cells, that may be cultured into a whole plant. Germplasm resources provide sources of genetic traits used by plant breeders to improve commercial cultivars.

Plant: Plant means without limitation, cells with or derived from, for example plant seeds, plant tissue suspension cultures, plant tissues, plant tissue explants, plant embryos, plant parts, meristematic tissue, callus tissue, leaves, roots, shoots, gametophytes, sporophytes, pollens, microspores and like.

Plant Parts: Plant parts include but not limited to leaves, stems, buds, roots, root tips, anthers, seed, grain, embryo, pollen, ovules, flowers, cotyledons, pods, tissue culture, cell culture, or any biological material derived from the plant like nucleic acid, protein etc. and like.

Herbicides: Herbicides are the chemicals that are used to kill unwanted plants. Here herbicides include herbicides that have mode of action of an HPPD inhibitor. Reference to a herbicide may include one or more herbicides. This include but not limited to tolpyralate ‘fenquinotrione, mesotrione, tefuryltrione, isoxaflutole, pyrasulfotole, benzobicyclon, benzofenap, pyrazolynate, pyrazoxyfen, bicyclopyrone, sulcotrione, tembotrione, topramezone.

Sequence identity: sequence identity is the percentage of nucleotides or amino acids that is identical between two sequences after proper alignment of those sequences. The person skilled in the art is aware of how to align sequences. To obtain the most significant result, the best possible alignment that gives the highest sequence identity score should be obtained. The sequences are compared over the length of the shortest sequence in the assessment.

Wild-type: A wild type plant as referred to herein is a plant which does not comprise a mutation of the invention, or which would be killed or have their viability significantly affected by an HPPD herbicide.

Alternate Codons or Alternative Codons: Refer to the sequences that comprises different codons that codes for the same mutation. Alternate codons or alternative codons for the mutation of the invention are shown in SEQ ID NOs: 14 to 18 (where the codon is shaded and underlined).

The present invention is in particular directed towards a plant that show enhanced resistance or tolerance to an HPPD herbicide, such as one or more of tembotrione, mesotrione, byciclopirona, tolpyralate, isoxaflutole and topremazone, to improve weeds control in the crop field compared to a wild-type plant.

The HPPD inhibitor herbicide, may be selected from benzoylpyrazole herbicides, benzoylcyclohexanedione herbicides, aroylcyclohexanedione herbicides, oxazole herbicides, cyclopropylisoxazole herbicides, pyrazolone, carbobicyclic compounds, cyclic ketone compounds, benzoylpyrazole herbicides, pyrazole herbicides, triketone herbicides, aroylcyclohexanedione herbicides. The HPPD inhibitor herbicide is in particular selected from tolpyralate, fenquinotrione, mesotrione, tefuryltrione, isoxaflutole, pyrasulfotole, benzobicyclon, benzofenap, pyrazolynate, pyrazoxyfen, bicyclopyrone, sulcotrione, tembotrione, topramezone or combination thereof.

The present invention is also directed towards the novel and distinctive allele and mutation designated as ADV-HT2, that confers non-transgenic resistance to the said HPPD herbicides in a plant and encode polypeptide having or comprising SEQ ID NO. 3.

The ADV-HT2 allele is found in a region of chromosome 3. A nucleic acid sequence encoding the mutant allele is provided herein as SEQ ID NO: 1, SEQ ID NO: 11 and SEQ ID NO: 13 to 18. These sequences encode a protein comprising a tryptophan to serine substitution at position 445 of SEQ ID NO: 3. A non-mutated sequence (also referred to as a wild type sequence) which encodes a protein having a tryptophan amino acid at a position corresponding to position 445 is encoded by SEQ ID NO: 2 or 12. Preferably, the ADV-HT2 allele is located at position 5283 of SEQ ID NO: 1 and may encode a polypeptide of SEQ ID NO: 2 in the wild type form or a sequence of polypeptide SEQ ID NO: 3 in the mutated form. The ADV-HT2 allele of the invention may comprise any one of the alternate codons as defined herein, each of which encode a serine residue. The alternate codons which encode a serine residue at position 445 of a protein of SEQ ID NO: 3 are shown in SEQ ID NO: 14 to 18 (underlined and shaded). Included within the scope of the invention is a nucleic acid sequence encoding a protein of SEQ ID NO: 3 or a protein substantially identical thereto, wherein the nucleic acid sequence comprises one of alternate codons 1 to 5.

In order to obtain herbicide-tolerant plants, endogamic sorghum (Sorghum bicolor) line BV94-4045 (Advanta proprietary elite line) seeds were treated with an aqueous solution of ethyl methanesulfonate (EMS). Treated seeds were planted and self-pollinated for advancing generation. Eight hundred nighty five M1 plants were selected, and two seeds of each plant were planted in a nursery, thereby obtaining a total of 1790 M2 plants. Pollen from both plants of each pair were collected, and the bulk was used for pollinating both plants of the pair. A bulk of M3 seeds from each pair was obtained from the 895 pollinated M2 pairs of plants. A total of 895 furrows were planted with the M3 progeny. Fifty plants from each M3 furrow were sprayed with 33.6 ml active ingredient /ha of topramezone. Six plants from the furrow number VT09-8800 showed normal growth and absence of symptoms after the treatment with herbicide and were considered as resistant to the herbicide and identified as BVT09-8800-1, BVT09-8800-2, BVT09-8800-3, BVT09-8800-4, BVT09-8800-5, and BVT09-8800-6. The genealogy of the resistant plants from the furrows was identified and they were designated BV94-4045EMS1-256-2 (hereinafter referred to as ADV-HT2). Herbicide tolerant M7 mutant plants and seeds selected from the original ADV-HT2 mutant, designated as: Material ID=VT10-11924-1-BK and Inbred Code=BV10-11924, were obtained and deposited with the NCIMB collection with Access No. NCIMB 43919 under the terms of the Budapest Treaty,

The present invention is not limited to sorghum plants mutated with EMS. Within the scope of the present invention are plants, for example sorghum plants, obtained by other mutation methods, for example methods such as CRISPR Cas, radiation and chemical mutagens, or by breeding or transgenic methods as described herein. Herbicide-resistant mutant plants can also be obtained by means of a process of selective pressure on cells cultured with a herbicide and selection of resistant cells to generate a herbicide-resistant plant. Details of mutation and breeding methods can be found in “Principles of Cultivar Development” Fehr, 1993, Macmillan Publishing Company, the disclosure of which is included herein by reference.

The present invention includes within its scope any plant (including plant part, tissue, or seed) comprising the novel mutation of the invention, as described herein, wherein the mutation confers HPPD tolerance or resistance on the plant. A plant of the present invention is preferably a cultivated plant having improved agronomic characteristics that make it suitable for commercial cultivation. Suitably, a plant, plant part, tissue, or seed as described herein may be a sorghum plant, plant part, tissue, or seed.

For the present invention, the terms “herbicide-tolerant” and “herbicide-resistant” are used interchangeably and are intended to have an equivalent meaning and an equivalent scope. Similarly, the terms “herbicide-tolerance” and “herbicide-resistance” are used interchangeably and are intended to have an equivalent meaning and an equivalent scope. Likewise, the terms “HPPD-resistant” and “HPPD-resistance” are used interchangeably and are intended to be of an equivalent meaning and an equivalent scope as the terms “HPPD-tolerant” and “HPPD-tolerance” respectively. By “resistance or resistant” or “tolerance or tolerant” is meant that a plant is able to survive the effects of the herbicide, and show enhanced growth and survival compared to a control variety known to be susceptible to an HPPD herbicide. By susceptible is meant that the herbicide in an effective amount has one or more of the following effects: chlorosis, necrosis, and growth retardation, death of the plant. Depending on the susceptibility of a plant, a plant may be scored as 1 to 5, as set out in Example 2.

A plant of the invention may comprise a wild type HPPD gene. The HPPD gene is located in chromosome no. 2 or chromosome no. 4 of the Sorghum plant.

A plant of the invention may comprise nucleic acid having at least 85%-99% identity with SEQ ID NO:1. Suitably, the nucleic acid sequence encodes a protein of SEQ ID NO: 3 or substantially identical thereto and having a serine residue at a position corresponding to position 445 of SEQ ID NO: 3. Suitably, the nucleic acid comprises a mutation as defined herein, encoding a serine residue at the position corresponding to position 445 of SEQ ID NO: 3.

A plant of the invention may encode a protein sequence having at least 85%-99% identity with SEQ ID NO: 3 and comprising a Serine residue at position 445 of SEQ ID NO: 3. More preferably the nucleic acid present in the plant possesses at least 95% identity to SEQ ID NO:1, SEQ ID NO: 11 or SEQ ID NO: 13 to 18 and codes for a protein having at least 95% identity with SEQ ID NO: 3 and comprising a Serine residue at the position 445 of SEQ ID NO: 3.

A plant of the invention may comprise any one of the following codons in the nucleic acid sequence on chromosome 3, which encodes a protein of SEQ ID NO:3 or a protein substantially identical thereto: TCG, TCT, TCC, TCA, AGC, or AGT. These alternate codons each encode a serine residue.

A plant of the invention comprises a tryptophan to serine substitution at position 445 of the protein sequence of SEQ ID NO: 3. A plant of the invention may comprise a nucleic acid sequence encoding said protein.

A plant of the invention may be used in a method of plant breeding.

The mutation of the invention may be introduced from another plant which comprises the mutation of the invention, through commonly used breeding techniques, such as crossing and selection, when the plants are sexually compatible. Alternatively, the mutation of the invention may be introduced into a plant using a transgenic approach. Suitable techniques include for example an Agrobacterium-mediated transformation method, or a genome editing method such as homologous recombination or the use of a CRISPR/Cas system.

Such introduction can be from a plant of the same species, that usually can be crossed easily, or from a plant of a related species. Difficulties in crossing can be overcome through techniques known in the art such as embryo rescue, or cis-genesis can be applied. Suitably markers are used to follow the incorporation of the allele or QTL, into another plant.

In one of the embodiments, the method of producing the plant that comprises resistance or tolerance to HPPD herbicide comprises cross-pollination of a first plant with a second plant so as to produce hybrid seeds. The first and second plants may be sorghum plants. The first and second plants may be the same species or may be different species. The method may comprise growing a plant resulting from the cross, allowing the plant to grow fruit, and harvesting seeds from the fruit. The seeds produced can be sown and allowed to grow into a hybrid plant, particularly an F1 hybrid plant.

A method of producing a plant that comprises resistance or tolerance to HPPD herbicide as described herein may comprise one or more rounds of selfing, and/or crossing a plant from the F1 to generate a further generation.

A method of producing a plant that comprises resistance or tolerance to HPPD herbicide as described herein may further comprise crossing an F1 hybrid plant with a plant having another desired characteristic; and selecting desired backcross progeny. Other desirable can be selected from, but is not limited to, the following group: resistance to bacterial, fungal, or viral diseases, insect or pest resistance, improved germination, plant size, plant type, water stress and heat stress tolerance, and male sterility.

In an embodiment, both hybrid's parent plants comprise in their genome at least one copy of allele of the novel mutation, as defined herein. Preferably, both hybrid's parent sorghum plants are homozygous for a mutation of the ADV-HT2 allele as defined herein, which encodes a serine residue at a position corresponding to position 445 of SEQ ID NO: 3. Both parent plants may comprise a mutation in the nucleic acid sequence on chromosome 3 as defined herein. In an embodiment, the nucleic acid sequence may comprise a C residue at position 5283 of SEQ ID NO: 1 or has alternate codons that code for a serine residue at position 445 of SEQ ID NO:3. Alternate codons 1 to 5 are shown in SEQ ID NOs: 14 to 28 (underlined and shaded)

In an embodiment, the first sorghum plant comprises in its genome at least one copy of an allele of the novel mutation as defined herein. The first sorghum plant may comprise a sequence encoding a protein of SEQ ID NO: 3 or a protein substantially identical thereto having a serine residue at a position corresponding to position 445 of SEQ ID NO: 3. The allele may be one of the alternate codons defined herein.

In one preferred embodiment of the invention, a hybrid plant of the invention is produced by crossing two plants heterozygous or homozygous for an allele of the novel mutation as defined herein. The hybrid plant may comprise a nucleic acid sequence encoding a protein of SEQ ID NO: 3 or a protein substantially identical thereto having a serine residue at a position corresponding to position 445 of SEQ ID NO: 3. The allele of the nucleic acid sequence may be one of the alternate codons defined in Alternate Codons. All of the resulting hybrid seeds and hybrid plants grown from such seed are expected to comprise in their genomes the ADV-HT2 mutation as defined above.

In one preferred embodiment of the invention, a hybrid plant of the invention is produced by crossing two plants homozygous for mutation of the ADV-HT2 allele as defined above.

In one preferred embodiment of the invention, a hybrid plant of the invention is produced by crossing two plants heterozygous for a mutation of the ADV-HT2 allele as defined above.

In one preferred embodiment of the invention, a plant produced is heterozygous for a mutation of the ADV-HT2 allele, as defined above.

In one preferred embodiment of the invention, a plant produced is homozygous for a mutation of the ADV-HT2 allele as defined above.

For the purposes of the present invention unless otherwise expressly indicated or apparent from the context, a “progeny plant” is any plant that is descended from at least one plant of the invention and includes, but is not limited to, first, second, third, fourth, fifth, sixth, seventh, eighth, ninth, tenth or any generation descendants of the plant of the invention. Preferably, such progeny or descendants comprise increased resistance to at least one HPPD herbicide when compared to a wild-type plant and such progeny or descendants further comprise an ADV-HT2 mutation as defined herein, and preferably as present in accession number NCIMB 43919.

The method of producing a hybrid or inbred plant comprising the novel ADV-HT2 mutation as defined herein may further involve growing a seed resulting from such crossing and selecting for at least one progeny plant with tolerance or resistance to HPPD herbicide. This selection may be made by applying the HPPD herbicide to a population and selecting any tolerant or resistant plants. Such progeny can be also selected using genetic methods, such as PCR amplification, to determine the presence of ADV-HT2 mutation as defined herein.

The present invention provides plants, or plant part, tissue or seed with increased resistance or tolerance to at least one herbicide, particularly an HPPD herbicide. The preferred amount or concentration of the herbicide is an “effective amount” or “effective concentration.” By “effective amount” and “effective concentration” is intended an amount and concentration, respectively, that is sufficient to kill or inhibit the growth of a similar plant, plant tissue, plant cell, or host cell, but that said amount does not kill or inhibit as severely the growth of the herbicide-resistant plants, plant tissues, plant cells, and host cells of the present invention. Typically, the effective amount or effective concentration of a herbicide is an amount or concentration that is routinely used in agricultural production systems to kill weeds of interest. Such an amount is known to, or can easily be determined by, those of ordinary skill in the art. The sorghum of the present invention comprise commercially acceptable levels of resistance or tolerance of HPPD herbicide. The effective amount or concentration that is routinely in the fields or otherwise to kill a weed or weeds of interest.

The herbicide resistant or tolerant plants of the present invention find use in the methods for controlling weeds. Thus, the present invention further provides a method for controlling weeds in the vicinity of the herbicide resistant or tolerant plant.

For management of weeds in the vicinity of herbicide tolerant or resistant sorghum plant of present invention a wide variety of formulations can be employed for protecting plants from weeds to enhance plant growth and reduce competition of nutrients. A herbicide can be used for pre-emergence or post-emergence or pre-planting or at time of planting to control the weeds. The herbicide can be present with different additives. The herbicide may also be used as the seed-treatment. Additives present in the herbicide formulation may include but not limited to other herbicides, detergents, adjuvants, spreading agents, sticking agents, stabilizing agents, or the like. The formulation can be a wet formulation or a dry formulation or any other suitable form like suspension concentrate, emulsion concentrate, encapsulated, non-encapsulated and others.

A herbicide may be applied at the recommended concentration, or at a concentration of 0.5×-5×, preferably in the range of 0.5×-4×, more preferably in the range of 1×-4× of the recommended dose.

The herbicide formulations can be applied in accordance with the conventional methods for example by spraying, dusting, irrigation and like.

The present invention provides non-transgenic and transgenic seeds with increased tolerance to any HPPD herbicide than the wild type. Suitably, a seed of the invention is a sorghum seed. Suitably, a seed of the invention has the ability to grow into a plant. Suitably, a plant grown from a seed of the invention has tolerance or resistance to an HPPD herbicide.

The present invention provides a diagnostic method to identify the presence or absence of the novel mutation present on Chromosome 3 of sorghum plant that imparts HPPD herbicide tolerance or resistance to the plant. Any suitable method may be used. Suitable diagnostic methods are discussed below that can be used to detect the novel mutation in a commercial or experimental plant or its parts. Also, such methods can be used to confirm any sequence comprising the given mutation defined herein. A suitable method may comprise detection of an allele which is in linkage with a mutation of the invention, for example detection of a mutation on chromosome 3, comprising a G to C substitution at position 51 of SEQ ID NO: 8.

A suitable method for determining the presence or absence of a mutation present on Chromosome 3 of a plant that imparts HPPD herbicide tolerance or resistance to the plant, or a mutation in linkage with a mutation of the invention as defined herein, may comprise PCR.

A “primer” is a single-stranded oligonucleotide, having a 5′ end and a 3′ end, that is capable of annealing to an annealing site on a target DNA strand, and the primer serves as an initiation point for DNA synthesis by a DNA polymerase, particularly in a polymerase chain reaction (PCR) amplification. Such a primer may or may not be fully complementary to its annealing site on the target DNA.

An “annealing” site on a strand of a target DNA is the site to which a primer is capable of annealing in the methods of the present invention.

Generally, for the amplification of a fragment of a gene by PCR, a pair of primers that anneal to opposite strands of a double-stranded DNA molecule are employed. By standard convention and used herein unless otherwise indicated or apparent from the context, the “forward primer” anneals to the non-coding strand of the gene and the “reverse primer” primer anneals to the coding strand.

The invention involves the use of a number of PCR amplifications. These primers are disclosed below:

Primer 1:
(SEQ ID NO: 5)
GAAGGTGACCAAGTTCATGCTGGAGGGCGCCATCTACCTCTC
Primer 2:
(SEQ ID NO: 6)
GAAGGTCGGAGTCAACGGATTGAGGGCGCCATCTACCTCTG
Reverse primer:
(SEQ ID NO: 7)
GTTGGAGCAGTTGTCCGGCAGT

In a preferred embodiment the forward primer and reverse can comprise of any nucleotides length that ranges between the nucleotide number of SEQ ID NO: 1.

In a preferred embodiment, the forward and reverse primers can span in the region of Seq ID no. 11. Preferably to detect the mutation, at least one of a pair of primers or probes contains the mutated nucleotide of the invention.

In another preferred embodiment, one of a pair of primers or probes for determining the presence or absence of the mutation may span the region of sequence of SEQ ID NO: 8, where the presence of the “C” at position 51 of SEQ ID NO: 8 the allele is indicative of HPPD-type herbicide resistance.

(SEQ ID NO: 8)
CCCGCTCGGTGAGGACAATGTGAAGGGCGGGGAGGGCGCCATCTACCTC
T[C/G]GGCCAAACTGCCGGACAACTGCTCCAACGATTTTGAAGTTGTC
AGGTGGC

In another preferred embodiment, one of a pair of primers or probes for determining the presence or absence of the mutation may comprise the sequences that detect one of the Alternate Codons as defined herein.

Primers suitable for use in the present invention may not be 100% complementary to a sequence provided herein including SEQ ID NO:1, 11 or 8, but will have the ability to bind thereto in a manner which distinguishes between the wild type and mutant forms of the ADV-HT2 allele. The allele of the invention is located at position 1001 of SEQ ID NO: 11. A suitable primer or probe may therefore bind under stringent conditions, which will be known to a person skilled in the art.

The results of PCR can be detected using KASP genotyping techniques and other standard used techniques including but not limited to gel electrophoresis, fluorescence assays.

A method for determining resistance of a plant to HPPD resistance may comprise determining the presence or absence of a QTL associated with HPPD resistance. Such a QTL may be a sequence comprising or consisting of a sequence provided herein, for example SEQ ID NO: 1, 11 or 8, or a sequence having at least 75%, 80%, 85%, 90%, 95%, 96%, 97%, 98% or 99% sequence identity thereto. Such a QTL may comprise a mutant allele of the invention. The method may subsequently comprise determining the presence or absence of a mutation of the invention within said QTL.

A method for determining resistance of a plant to HPPD resistance may comprise determining the presence or absence of a molecular marker associated with HPPD resistance. Such a molecular marker may be a genetic marker. Such a marker may be a mutation as described herein. Such a method may be performed in combination with one or more diagnostic methods as described herein.

In addition to PCR amplification, the methods of the invention can involve various techniques of molecular biology including, for example, DNA isolation, particularly genomic DNA isolation, digestion of DNA or PCR products by restriction enzymes and nucleases, DNA ligation, DNA sequencing, agarose gel electrophoresis, polyacrylamide gel electrophoresis, gel electrophoresis in any other suitable matrix for the electrophoretic separation of DNA, the detection of DNA by ethidium-bromide staining, and the like. Such techniques are generally known in the art and are disclosed, for example, in Sambrook et al. (1989) Molecular Cloning: A Laboratory Manual (2d ed., Cold Spring Harbor Laboratory Press, Plainview, N.Y.).

For the present invention, genomic DNA of the plant can be isolated from whole plants or any part, organ, tissue, or cell thereof. For example, genomic DNA can be isolated from seedlings, leaves, stems, roots, inflorescences, seeds, embryos, tillers, coleoptiles, anthers, stigmas, cultured cells, and the like. Furthermore, the invention does not depend on the isolation of genomic DNA from plants or parts, organs, tissues, or cells thereof that are of any particular developmental stage. Furthermore, the invention does not depend on plants that are grown under any particular conditions. The plants can be grown, for example, under field conditions, in a greenhouse, or a growth chamber, in culture, or even hydroponically in a greenhouse or growth chamber.

Typically, molecular markers are detected by any established method available, including, without limitation, allele specific hybridization (ASH), real-time PCR assays for detecting single nucleotide polymorphisms (SNP), amplified fragment length polymorphism (AFLP) detection, amplified variable sequence detection, randomly amplified polymorphic DNA (RAPD) detection, restriction fragment length polymorphism (RFLP) detection, self-sustained sequence replication detection, simple sequence repeat (SSR) detection, single-strand conformation polymorphisms (SSCP) detection, isozyme markers detection, or the like.

In another embodiment the results of the mutation can also be assessed using protein assays, protein probes or different antigen detection techniques including but not limited to ELISA.

The present invention also provides kits for performing the methods for genotyping a plant comprising an ADV-HT2 mutation, or an allele in linkage with a mutation of the invention. Such kits may comprise one or more sets of forward and reverse primers for amplifying a region of chromosome 3, for example comprising the ADV-HT2 allele or a region comprising SEQ ID NO: 8. For example, a kit may comprise one or more sets of primers for example as defined herein for amplifying a region of a nucleic acid sequence provided herein, including for example SEQ ID NO: 1, 11 or 8.

In preferred embodiment the forward and reverse primers can be of any length and span in the region of any of the nucleic acid sequences provided herein. Suitably, the forward and reverse primers may be of any length and span in the region of SEQ ID NO: 11. Preferably to detect the mutation at least one of the primer or probe designed contains the mutated nucleotide of SEQ ID NO: 1, 11, or 13 to 18.

More preferably the primers are selected from below list

Primer 1:
(SEQ ID NO: 5)
GAAGGTGACCAAGTTCATGCTGGAGGGCGCCATCTACCTCTC
Primer 2:
(SEQ ID NO: 6)
GAAGGTCGGAGTCAACGGATTGAGGGCGCCATCTACCTCTG
Reverse primer:
(SEQ ID NO: 7)

In preferred embodiment the forward primer and reverse can comprise of any nucleotides length that ranges between the nucleotide number of SEQ ID NO: 1.

In preferred embodiment the forward and reverse primers can span in the region of SEQ ID NO: 11. Preferably to detect the mutation at least one of the primer or probe designed contains the mutated nucleotide of the invention.

In another preferred embodiment the primers or molecular markers for detecting the mutation span in the region of sequence with SEQ ID NO: 8, where the “C” allele is indicative of HPPD-type herbicide resistance

(SEQ ID NO: 8)
CCCGCTCGGTGAGGACAATGTGAAGGGCGGGGAGGGCGCCATCTACCTC
T[C/G]GGCCAAACTGCCGGACAACTGCTCCAACGATTTTGAAGTTGTC
AGGTGGC

The kits can optionally comprise one or more of the following: a polymerase, deoxyribonucleotide triphosphates, any preferred reagent, a label, and instructions for performing the method.

The kits may further comprise a manual comprising instructions to carry out the method to detect the ADV-HT2 mutation in the given sample.

The mutation of the present invention may be used in the identification of further markers for HPPD tolerance or resistance, for example markers which are in linkage disequilibrium with the mutation of the invention and which have a phenotype of tolerance or resistance to and HPPD herbicide.

### EXAMPLES

The following examples are illustrated by the way of illustration and not by the way of limitation. It should be clearly understood that other embodiments, modifications and equivalents of the invention may be possible after reading the present description.

### Example 1: Development of Mutant Sorghum Lines and Mutant Allele ADV-HT2 Mutagenesis of Endogamic Sorghum Line BV94-4045 (Advanta) and Selection of HPPD Moa-Resistant Mutant BV94-4045EMS1-256-2

Thirty-two thousand pre-germinated sorghum seeds from endogamic line BV94-4045 were dipped into an aqueous solution of 0.05% v/v ethyl methanesulfonate (EMS) for 16 hours. Treated seeds were planted at the Experimental Station of Advanta Seeds in Venado Tuerto, Province of Santa Fe, Argentina, plot No. 1, and they were self-pollinated for advancing endogamic generation to M1.

A total of 895 M1 plants were selected and two seeds from each plant were planted at the Experimental Station of Advanta Seeds in Oran, Province of Salta, Argentina in a nursery and a total of 1790 M2 plants was obtained. Pollen from both plants of each pair were collected, and the bulk was used for pollinating both plants of the pair. A bulk of M3 seeds from each pair was obtained from each of the 895 pollinated M2 pair of plants. A total of 895 furrows of M3 progeny were planted, in Venado Tuerto and 50 plants from each M3 furrow were sprayed with 33.6 ml active ingredient/ha of topramezone. Six plants from the furrow VT09-8800 showed normal growth and absence of symptoms (no chlorosis nor necrosis) after treatment with herbicide and were considered resistant to the herbicide and identified as BVT09-8800-1, BVT09-8800-2, BVT09-8800-3, BVT09-8800-4, BVT09-8800-5, and BVT09-8800-6. The genealogy of the resistant plants from the furrows was identified and they were designated BV94-4045EMS1-256-2 (hereinafter referred to as ADV-HT2).

### Example 2: Genetic Screening of Sorghum Line Containing Mutation and Evaluation of HPPD Gene in Such Lines

### Step A) Trait Genetic Mapping

The main objective of the analysis was to genetically map (QTL map analysis) the resistance to HPPD-herbicide. Three different populations of seeds obtained from resistant plants of Example 1 were analyzed. Phenotypic evaluations on 3 mapping populations and parental lines were done at 8 and 20 days after HPPD-herbicide spraying (topramezone, Convey™, 1×). The plants response to herbicide was scored under two qualitative ordinal scales: a) measure with 5 scores (1: severe chlorosis and necrosis; 3: severe necrosis and growth retardation 5: necrosis and growth retardation; 7: mild necrosis; 9: no damage lines); b) measure with two scores (1: susceptible and 9 resistant). A consensus map was built using the JoinMap software. The QTL was located at the beginning of chromosome 3. The QTL region was 11 cM in size.

### Fine Mapping of the QTL Identified in Step A)

In order to narrow down the region responsible for the phenotype, the initial QTL region was sequenced to identify putative mutations. Inbred line BV10-11924 and also the line used for EMS treatment (BV94-4045) were sequenced in a Novaseq 6000 sequencer (S2 150 PE, ˜80× deep). All the variations were called in public reference genome Sbicolor_454_v3.0.1.fa.gz.

Using markers for the mutations found, a fine mapping analysis was done. Complete linkage disequilibrium of the present mutation in SEQ ID NO: 8 to ADV-HT2 phenotype was observed within two F2:3 mapping populations.

From the analysis it was found that there is no evidence of effect of the SNPs in SEQ ID NO: 9 or Seq. ID No. 10 on the phenotypic scores, while evidence of the effect of SEQ ID NO: 8 (FIG. 6) with the Allele in SEQ ID NO: 1.

(SEQ ID NO: 9)
CGAACAGTACAATCAGCCATGCGCTGTGTGTCCTCAACTCCACTGACTT
C[A/G]CAATCAAAGAACCCCATGGACATGCATGTCATTCCGGATCTGT
GCAGCGA
(SEQ ID NO: 10)
TCACATCGAATCTTGTGGCACATGCATTAAGCATTAAATATAGTCGAAA
A[T/C]AAAAACAAATTGCACAGTTGCCTGTAAATCGTGAGATGAATCT
TTTGAGC

The mutation in SEQ ID NO: 8 is the only mutation in chromosome 3 between the mutations in SEQ ID NO: 9 and SEQ ID NO: 10.

Analysis of HPPD Genes in the Mutated Plant with HPPD Herbicide Resistance

In the sequencing analysis mentioned in step b) all HPPD genes were fully sequenced in both sequenced lines, and no-mutation was found in them when compared with reference to publicly available genome Sbicolor_454_v3.0.1.fa.gz.

There are 3 homologous genes in Sorghum that may show 4-hydroxyphenylpyruvate dioxygenase activity (their functional annotation):

1) Sobic.004G053700 4-hydroxyphenylpyruvate dioxygenase. In chromosome 4.

2) Sobic.002G104200 4-hydroxyphenylpyruvate dioxygenase. In chromosome 2.

3) Sobic.002G105600 4-hydroxyphenylpyruvate dioxygenase. In chromosome 2.

### Results

The DNA sequence of the 3 HPPD genes showed no differences in line with novel ADV-HT2 when compared with the control line (BV94-4045, the line used for EMS treatment). Showing the public wild type sequence published in Sbicolor_454_v3.0.1.fa.gz.

From the above it is confirmed that that no conventional mutation is responsible for ADV-HT2 phenotype other than the mutation in SEQ ID NO: 1.

### Example 3: Field Testing of Mutant Sorghum Line Containing Mutant Allele ADV-HT2

To determine the spectrum of tolerance to different HPPD herbicide site of action (SOA) active ingredients (AI) in a sorghum HPPD tolerant inbred BV10-11924 (ADV-HT2) following methodology was used.

Two grain sorghum genotypes were evaluated under field conditions with herbicide pre-emergency (PRE) and post-emergency (POE) treatments of 6 active ingredients of HPPD SOA herbicide families for sorghum crop phytotoxicity trials.

The experiment design was a split-plot design with 2 replicates where: Herbicide treatment was defined as Main plot, and Genotype as split plot. Plot size were 10 square meters each, this is 4 row/plot, 5 meters length and 0.52 meters between rows.

Both genotypes (Table 1) were sown in the Advanta R&D Station in Venado Tuerto, Argentina using an experimental planter. Wild type BV94-4045 (line used for mutagenesis in example 1) is a maintainer elite inbred line Advanta's proprietary.

All herbicide treatments (Table 2) were applied same day of sowing date for herbicide PRE treatments, and 15 days after sowing for POE treatments using an experimental sprayer in optimal weather conditions. The herbicides rates were defined as: 1×, 2× and 4×. Along with the herbicides, there were applied specific surfactant when they were recommended by the supplier for each herbicide. In all cases, 1× rate was defined based on the recommended dose in herbicide label.

Two weeks after spraying (WAS), phytotoxicity (plants response to the herbicides) was evaluated using Aerial Biomass as % Control (Biomass % Control) as an indicator for herbicide phytotoxicity parameter.

For Aerial Biomass (Biomass) measurements, all plants from two central rows each plot were harvested by cutting them at ground level and weighted before and after drying at 60° C. for 48 h in order to determine the shoot dry mass reduction of each treatment. All data were standardized using the Aerial Biomass average as the % of Control plots. For the calculation as the percentage of its respective untreated control was used the following function:

Biomass % Control=(Biomassgh/Biomassg Control)*100

where “Biomass % Control” is the Aerial Biomass expressed as percent of each genotype in the Control plot without any herbicide application (Table 3); “Biomassgh” is the Aerial Biomass is the mean for each genotype*herbicide treatment combination and “Biomassg Control” is the Aerial Biomass mean for each genotype in the treatment CONTROL (untreated).

TABLE 1
DETAILS OF GENOTYPES INCLUDED IN THE EXPERIMENT

| # | Material ID | Generation | Details |
|---|---|---|---|
| 1 | BV10-11924 | M7 | Mutant ADV-HT2 |
| 2 | BV94-4045 | Inbred | Susceptible wild type |

TABLE 2
DETAILS OF HERBICIDE TREATMENTS. LABEL
RECOMMENDED RATE IS CONSIDERED AS 1X.

| | | | | | Dose | |
|---|---|---|---|---|---|---|
| | | | Time of | | Commercial | |
| | | | Applica- | | Product per | |
| # | Code | Active Ingredient | tion | Rate | Hectare | |
| 1 | CONTROL | | | NA | | |
| 2 | TOP-1X | TOPRAMEZONE | POE | 1X | 100 | ml/ha |
| 3 | TOP-2X | | | 2X | 200 | ml/ha |
| 4 | TOP-4X | | | 4X | 400 | ml/ha |
| 5 | MES-1X | MESOTRIONE | POE | 1X | 300 | ml/ha |
| 6 | MES-2X | | | 2X | 600 | ml/ha |
| 7 | MES-4X | | | 4X | 1200 | ml/ha |
| 8 | ISO1-1X | ISOXAFLUTOLE | PRE | 1X | 150 | ml/ha |
| 9 | ISO1-2X | | | 2X | 300 | ml/ha |
| 10 | ISO1-4X | | | 4X | 600 | ml/ha |
| 11 | BYC-1X | BICYCLOPIRONA | PRE | 1X | 750 | ml/ha |
| 12 | BYC-2X | | | 2X | 1500 | ml/ha |
| 13 | BYC-4X | | | 4X | 3000 | ml/ha |
| 14 | TOL-1X | TOLPYRALATE | POE | 1X | 100 | ml/ha |
| 15 | TOL-2X | | | 2X | 200 | ml/ha |
| 16 | TOL-4X | | | 4X | 400 | ml/ha |

### Results

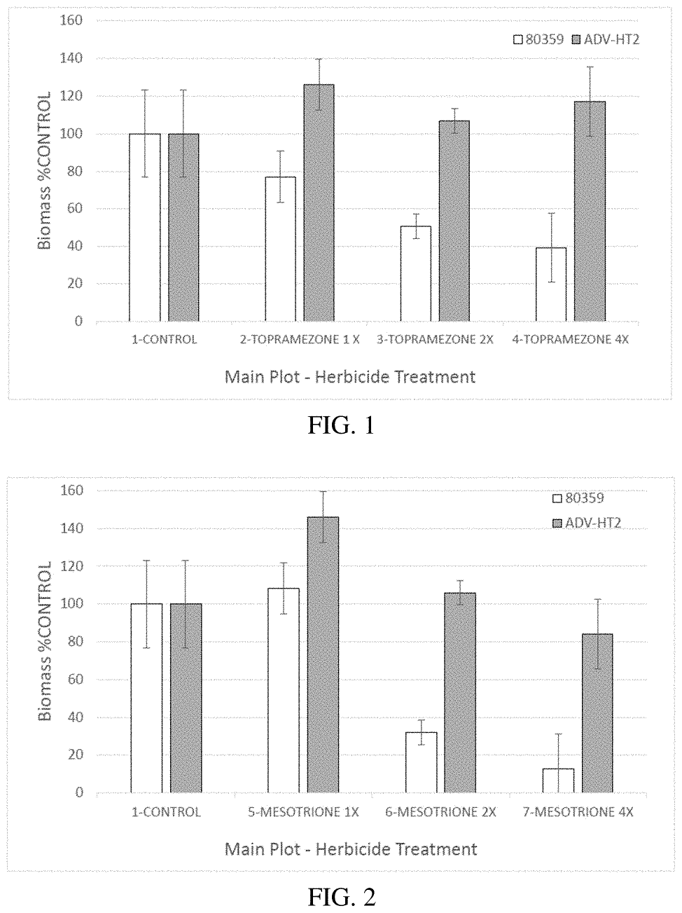

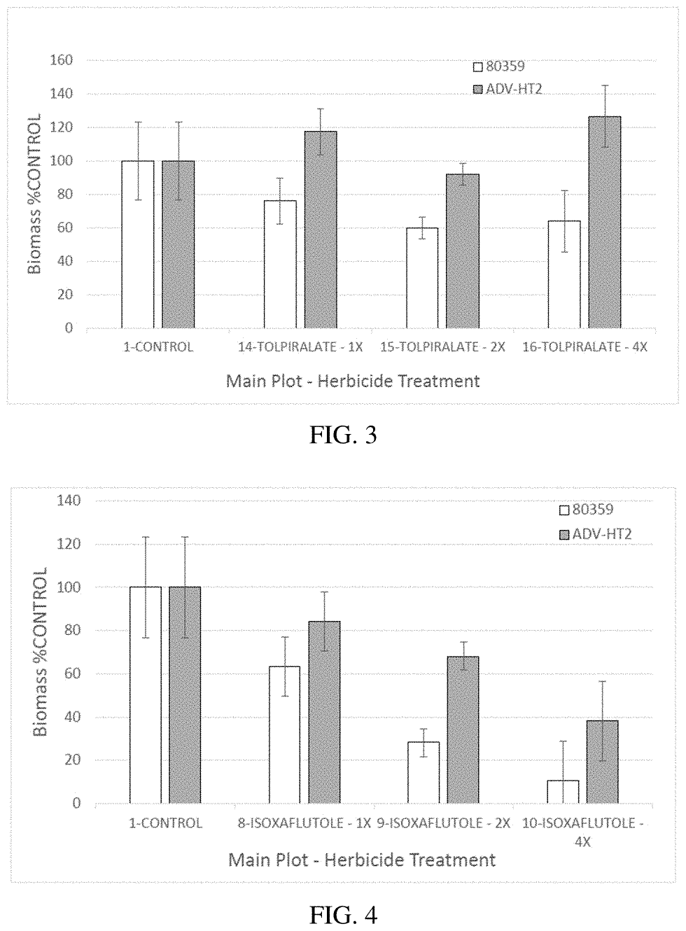

In the case of herbicide treatments with time of application in POE (TOPRAMEZONE, MESOTRIONE, TOLPIRALATE), wild type inbred line BV94-4045 was susceptible and showed a phytotoxicity with a significant reduction in the biomass accumulation 20 days after herbicide application for all herbicides and rates: Topramezone: 1×, 2×, 4×; Mesotrione: 2×, 4×; Tolpyralate: 1×, 2×, 4×, but Mesotrione 1× (Table 3, FIGS. 1, 2, and 3).

The genotype BV10-11924 (mutant ADV-HT2) was tolerant without phytotoxicity nor reduction in the biomass accumulation 20 days after herbicide application for all herbicides and rates combinations: Topramezone: 1×, 2×, and 4×; Mesotrione: 1×, 2×, and 4×; Tolpyralate: 1×, 2×, and 4×; (Table 3, FIGS. 1, 2, and 3).

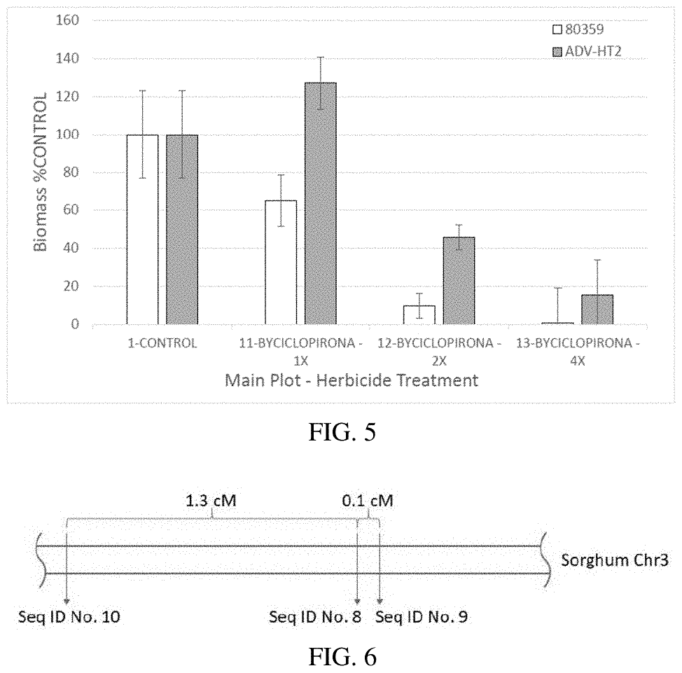

In the case of PRE herbicide treatments (ISOXAFLUTOLE and BYCICLOPIRONA), the mutant inbred line BV10-11924 (ADV-HT2) was tolerant, without phytotoxicity, for the 1× rate of herbicides with no significative reduction in biomass accumulation 20 days after the application. (Table 3, FIGS. 4 and 5).

TABLE 3
PHYTOTOXICITY OF TWO GRAIN SORGHUM PHENOTYPES,
BV94-4045 (WILD TYPE) AND BV10-11924 (MUTANT
ADV-HT2) TO 16 HERBICIDES TREATMENTS APPLIED
IN PRE OR POE, AS THE TOTAL BIOMASS ACCUMULATED
EXPRESSED AS A % OF BIOMASS ACCUMULATED IN
THE UNTREATED CONTROL TREATMENTS

| | |
|---|---|
| Herbicide | |
| Treatment | Biomass % Control |

| Code | BV94-4045 | BV10-11924 |
|---|---|---|
| CONTROL | 100 | 100 |
| TOP-1X | 77 | 126 |
| TOP-2X | 51 | 107 |
| TOP-4X | 39 | 117 |
| MES-1X | 108 | 146 |
| MES-2X | 32 | 106 |
| MES-4X | 13 | 84 |
| ISO-1X | 63 | 84 |
| ISO-2X | 28 | 68 |
| ISO-4X | 10 | 38 |
| BYC-1X | 65 | 127 |
| BYC-2X | 10 | 46 |
| BYC-4X | 1 | 15 |
| TOL-1X | 76 | 117 |
| TOL-2X | 60 | 92 |
| TOL-4X | 64 | 127 |

### CONCLUSION

It is evident from the above experiments that Novel ADV-HT2 mutation is not present in conventional HPPD gene and still confers HPPD herbicide resistance or tolerance in sorghum plant.

From the above field experiments it is also evident that sorghum plant with ADV-HT2 mutation is resistant and tolerant to a variety of HPPD herbicides at different concentrations and produces more yield than control.

The application can be fully understood from the following detailed description and the accompanying drawings and Sequence Listing. It should be noted that the nucleic acid and amino acid sequences listed in the accompanying sequence listing are shown using standard letter abbreviations for nucleotide bases, and one-letter code for amino acids. The nucleic acid sequences follow the standard convention of beginning at the 5′ end of the sequence and proceeding forward (i.e., from left to right in each line) to the 3′ end. Only one strand of each nucleic acid sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

(Nucleic acid sequence encoding protein of SEQ ID NO: 3 and
including a G to C substitution)
SEQ ID NO: 1
ATGGTTCACGGCGCCTCCGCGACAACGCAATTCATGCGCGCCGCGACGCAGCAGCA
GCAGCAAGGAAAGGACCGCGTGCCCGTGGGCGCCGGTCGACAAAAACCTCCTCGCC
CCCGGAGGGGCATCGGATCCAGCCGAACCGCAGCGTGTACGACCGCGGAGCGGCAA
GCGGCGCGACCCGAACCCGACGCGAGCTGCCCGGGCGCACCGGCAGTCCGGCCTCG
TGCTTCTGCATTGGGTCGCGATCCAAATCCTGGGAGGCTCTCCTGTCTTCTCTGGCTA
TCCGAACAGGTCCGTCCGAGCATTATTTGTCAGATCTCTTTCCGTCTTGTCAGTTGTC
CACGGTCTCCGAGCCCTCCTATTTGCATCCACGTAGATTGATGATAGATTCCTCTCGG
AAGTGTCTCGAAATTTTCTAAACTCTAAAATTAGAAAGTTAGTACTTGTGAAATTCT
TTAAAAAAAAAATTACTAGTAAAACTGAGCTGCGCTAAACAGATTTCGCCAGATTTG
AAGATCTGAGATTTCGAGATTATCTAGTGCAGAGGGACATTTCTTATTTTTTACCTTC
TCAAATTTGAAACACTAAACGACTTCTTTTTTCAGCAGGAGAGACTCAGATTCTGAT
CGCATTTTTTTTTCACTGATCACATTTGTGCTTCTCTCTCGTAGGCCTTGTTTAGTTCC
GAAAAGTGAAAACTTTTTGGAACTGTAGCATTTTCGTTTGTTTGATCATGGACTAAC
TAGGATCAAAAGATTCGTCTTGTGATTTACAGCTAAACTGTGTAATTAGTTTTTGTTT
TCGTTTATATTTAATGTTTCATGCATGTGACACAAGATTCGATGTGACGGGAAATCTT
GAAAACTTTTTGGTTTTCAGGGTAAACTAAACAAGGCCGTAGTCGTTCTGGACAAGT
TTTGTCCTAACGAGCAGAAGCGTACTGACGAAACTTGTCTTCACTGGCCCATTGGAT
CTTGTGGCAGCTCATGCAAAAAATATTTCGATAGTTGCCTATCACACGCATGTGTTT
ACTGTCCACAGACGCTGATTCTAAAGAATCTTTTCACCATCAATTGGCCGCGACATT
TTGGCGATTTCTCTGCGAGACTCACTGATGACACATTACTTTTGCATTTATTAAAAAA
TCCAATCTTTTTGTCTCTTTTTACCCGAAAAAAGCTCCCTCCTTTTTTGACCCCTCTTC
CTTCCCCTGAAACGCACGCAGACGCAGCTGAGCCTGAATTCTTATCCACCCACGGCG
CCACCCTCCACTCCCGGAACGTAAGCCAAAAGATCAGAGAAAGAGACAGACAGAC
AAGATTAAAAGAGTGACAAGGCAAAGAGAGAGAAAAAAGAGTAAGAAACCGCCCT
AGAATCTTTTCAGTTTTTCCCTGTCTTCATGTGTCTTTGTTATAGAGACGCTGCCTTGT
CGGGGCAGCAGACTTGGGACGAGAGAGAGGCCTAGAGGGGGAGGAGTTGGTGAGC
GAGCTCGGGATCAGCGGGTGACCCCAGAGGTTGTTGCCTCTGGAAGTTCCCAATTCT
TTAACCCCCATCTTCTTCCACATGACTTTGGACTCGCTGCTGTTCTTTTGGTTGGTAT
ATCCTTGGATCTCTGACTCCGCCTGTTCCCCGAAATTTCCAGTTCTTGCTTCCTCTTG
GAGGAGGGCTGCAGCCTGCACCCTGCAGGGGAGAGAGAGCAGGAGGGGACGGAGA
CATGGGTAGCTTCGCTAAGCTGGCGAAGAGGGCGGTGGAGACGGACGCGCCGGTCA
TGGTGAAGGTGACGAATTTCTCCTACCTCGTCGAAATCGCATCTTGACTCTGGAGTT
TCTTGGGCTTCCTTGGTGAGTTCTTTCTAATAAGACGTCCCTTTTTCAGATACAAGAA
CTGCTTCGAGGGGCCACGGATGTCATGTCGCTTGCGCAGGTATATGACCATAATTTT
CTCGATTTGGCTGTGCAATGGTTTCCTGCTTTGTGATGTTATTTAAGGTTAATGGAAC
AAGCACGAAACGGAAAGTCAAACGAAAGCAGAACGGATTAATGGGAGTCGATCAT
CTTATAGCAATTCGCTCCCAAATGGTTTCTGTCAATTGTTCAAGTTGAAATGTACTAC
TGTAGTTCGGTACAGTTCATTTGCTACGTTTCGTGTAACAAATCATACTGTTTTTACT
TGCTATTCCTGAATAGTTCTTATGTTGCCGAAGTGGGACATTCGTATGTAATCTAACT
AGTGACATGCGTGTCTTTTAGTGTCCTATTAGCATGACCCGACCATATGATATTTATT
TCTGAGAAATTCGTTTCTGTTCTTCACTTTTTGTAAATCCTGAATGCAGGGAGTTGTT
TACTGGCAACCTCCTGAGTCAGCTCTGGATAAGATCGAAAAAATCGTCAGGGAACC
AACAGTCAGTAAGTATGGTTCTGATGATGGACTTCCTGAGCTTCGAGAAGCACTTCT
CGAAAAGGTAACTTCTTTTTATCATAGAATATATAATATATTTTGCCCATACACTGAC
TAAAGCAGGCCATGCTCTTGAGTATTACCACATTGCCATGCCTTTGTCATTGGGAAC
TAGGGATCCTGCTTCTAAACCACTACACCGTAACCAAATTTTAATAGAGATATTTGA
TCTTAACTCACAGTCGCATCAGCAATCTCAAGTGACCGATGCAATGGTTCAGAAATT
ACTTTGTCAAAACAAAGGTTCCAAAAAATCTTTTTGTTCCCAGAGTTACATTCCTGG
GAAGCATCTAATTTTCTGAATATTGGCAGAGTGTGTCATAAAACTCTCTATTCATCA
ACTGAGAAATGGGAACAATATTCATTATTGTGAGTTTGTGACTCCTCTGGAAATTCA
GAAGAGAAATTCATGGTGGTGCTAAGAGTTGCAAAGTCTCTCTGCCATGTTGTTTGT
CTAACAGTTGAGATGAAATCCTGAGATGATTTAGGTGCTCTAACCGTTAGCCTAAAT
GCTCCACATCGCACAGTTATCTTTAGAATTTGTTCCACCGTTTTGTCACCTACTGGGC
TAAGATTTTTATTGCTGTTTGGACCACCAATGTGTTAAAATTAGTACTGCATTGCAAG
CATCTTGTATATCTTTCCTGATGTCTACTGTATTTTTGGCAGCTACGCAGAGAGAATA
AGCTTACCAAGTCATCAGTCATGGTCACTGCTGGTGCAAATCAGGTAAAAACTTACT
TTCCTATTTTGCCTTGTAAAGTTTTCAAACTGGGCCTTACGTTTCCCAATTTACTTATG
TTTATTACTTCTTTAGGGGCACACTCTTCCTTTCCTTTTTCACTGCTAGACCTTGAACT
TGTCTTACAATCTGGTCTTTTGTTACATTGATTTTTTCAGGCTTTTGTGAACTTGGTCC
TCACTCTTTGTGATGCTGGTGATTCTGTTGTCATGTTTGCACCATATTATTTCAATGC
CTACATGTCATTCCAGATGACAGGTGTTACTGACATATTAATTGGTGGTTGCGATCC
CAAGACACTTCATCCTGATGTTGGTAAGATGATTCTCTTCTTCTTTCTGTTTCATTGG
AGAAATTGGTGAGATGTCTTATATCAATAGTCTGTTGTAAGAAATTTGTTGTACTTA
GTGCTGGTTCAGGAGCATCTGCTTCATTGGCACATGTTCAATTTCCGTTATAAGTCTA
CATTATCTTTTAGCTGATGAGAAGATATGCTTTTGTAAAATACAACGCATCTAGTAT
AAGAAATTATGCATTGGACGTAGTTGATAATATTTCATTTTGGAAGCATACATTACA
ATTATGTCTAAAACTGCAGATTGGTTGGAGAAGGTTCTGAAAGAAAATGACCCTATC
CCTAAACTTGTTACTGTTGTGAATCCGGGGAACCCCTCTGGAGCTTTTGTTCCCAGGC
CTATGCTTGAGGTCATCTCTCTCCACTAACACTTGTGTTTGGAATCACTATCCAATAT
TTCCTAATATTGCATTGTCATTCCAGAGAATTTCAGATCTGTGCAAAAATGCTGGTG
CATGGCTTGTGGTTGACAATACCTATGAGTAAGTCAATACTGTAAAGTGATCATTTT
CACAAGCAATTATATAATCTTTTGTGGTATCTGTTAGAGAGAACTTAGAAGAGCCGG
GGAATTATGGTTGTAGTCAAAGGGACAGATCATATAAGCCATTAGAGCTCTTCTGCC
TCCTCTTGAATCTGTGTCCAGATGAAAGACACTCTGATGGCATATAGGATTTCTAGT
ATCTTTGCAGTTTCCACAAGGAAGGGAAAATCAGAATATTGTGCCCATAGGAGATTC
AGAAAGTAGTCGTCCTGCACAGATATGTTGTGTGAGAATATGTCGAATACAGTTATC
CTGTGTTTGTTTTTTTGGTTTAGGATAAGTTCTCTTATTCAAATTCATTCCAGCTGCCA
AGCCTTTATAAATACGTTTTAGGCTCTAAACTTGGTGATGTTAAAAATAAGCCCTCC
AATCACACTTACTGCTTTATACTTTATACAGGCCAGGGTTTATGTTTGCCTGAAAGA
GTCAGGTGTTCAGGTCCATGTTGAGGAACTAATAGTAATTAATAAAGGACCTTTTCC
CTCTAAGCTTAAAGAAACATACAAGTTTTTGTCGTGGAGCTGCGATTAGCCAACCAT
ATTTGAAGTGGTAGCATAAAGAATGGAGTCCAAACATGTCGAGTGGAACACAGAAA
GTGGCTTATTCCTTTTACTCCCTGCAAACGAATTCTGCTCCTTATTGGAATTTAGTTA
TGGTCTTTAGCATTTGAGCTTTGTAAGATTTGATGCTTAGGTCAGTTTTCCTGTGTCT
TTTTGCAGATACTTTATGTATGATGGAATGGAGCACTATTGCTTAGAAGATACTCAT
ATTGTCAACCTCTTCTCATTCTCAAAGGCTTATGGAATGATGGGGTGGCGTGTAGGA
TACGTGAGTGCATCATACTCTTCGTTTATCATTTTATTATGCTTTACTCCACTTTTTGG
CTGCTGCTGCTGTTAATTGTCACTGTAACAGTAAGTCATACATGGACTGTCTGCTGC
AGATTGCATTTCCAAATGAAGCTGATGGCTTCCACGATCAGCTCCTCAAAGTGCAAG
ACAACATACCTATCTGTGCCTCCATCATCGGGCAGCGCCTGGCGCTCTACTCACTGG
AGGCTGGCCCCGAGTGGATCAAAGAAAGGGTGAAAGACTTGGTGAAAAACCGAGC
ACTGCTCGTGGAGGCGCTGTCCCCGCTCGGTGAGGACAATGTGAAGGGCGGGGAGG
GCGCCATCTACCTCTCGGCCAAACTGCCGGACAACTGCTCCAACGATTTTGAAGTTG
TCAGGTGGCTTGCAAACAAGCACGGTGTCGCTGTGATCCCTGGCAGCGCCAGTGGA
GGCCCCGGATACATCCGCGTCTCCTTCGGAGGGCTCAAAGAAGAAGACACCAGGCT
CGCTGCTGAGAGGCTAAGGCGCGGCTTGCAGGAGCTGGTGACTGATGGAATGGTAC
AGTAA
(Wild Type nucleic acid sequence encoding protein of SEQ ID. No 4,)
SEQ ID NO: 2
ATGGTTCACGGCGCCTCCGCGACAACGCAATTCATGCGCGCCGCGACGCAGCAGCA
GCAGCAAGGAAAGGACCGCGTGCCCGTGGGCGCCGGTCGACAAAAACCTCCTCGCC
CCCGGAGGGGCATCGGATCCAGCCGAACCGCAGCGTGTACGACCGCGGAGCGGCAA
GCGGCGCGACCCGAACCCGACGCGAGCTGCCCGGGCGCACCGGCAGTCCGGCCTCG
TGCTTCTGCATTGGGTCGCGATCCAAATCCTGGGAGGCTCTCCTGTCTTCTCTGGCTA
TCCGAACAGGTCCGTCCGAGCATTATTTGTCAGATCTCTTTCCGTCTTGTCAGTTGTC
CACGGTCTCCGAGCCCTCCTATTTGCATCCACGTAGATTGATGATAGATTCCTCTCGG
AAGTGTCTCGAAATTTTCTAAACTCTAAAATTAGAAAGTTAGTACTTGTGAAATTCT
TTAAAAAAAAAATTACTAGTAAAACTGAGCTGCGCTAAACAGATTTCGCCAGATTTG
AAGATCTGAGATTTCGAGATTATCTAGTGCAGAGGGACATTTCTTATTTTTTACCTTC
TCAAATTTGAAACACTAAACGACTTCTTTTTTCAGCAGGAGAGACTCAGATTCTGAT
CGCATTTTTTTTTCACTGATCACATTTGTGCTTCTCTCTCGTAGGCCTTGTTTAGTTCC
GAAAAGTGAAAACTTTTTGGAACTGTAGCATTTTCGTTTGTTTGATCATGGACTAAC
TAGGATCAAAAGATTCGTCTTGTGATTTACAGCTAAACTGTGTAATTAGTTTTTGTTT
TCGTTTATATTTAATGTTTCATGCATGTGACACAAGATTCGATGTGACGGGAAATCTT
GAAAACTTTTTGGTTTTCAGGGTAAACTAAACAAGGCCGTAGTCGTTCTGGACAAGT
TTTGTCCTAACGAGCAGAAGCGTACTGACGAAACTTGTCTTCACTGGCCCATTGGAT
CTTGTGGCAGCTCATGCAAAAAATATTTCGATAGTTGCCTATCACACGCATGTGTTT
ACTGTCCACAGACGCTGATTCTAAAGAATCTTTTCACCATCAATTGGCCGCGACATT
TTGGCGATTTCTCTGCGAGACTCACTGATGACACATTACTTTTGCATTTATTAAAAAA
TCCAATCTTTTTGTCTCTTTTTACCCGAAAAAAGCTCCCTCCTTTTTTGACCCCTCTTC
CTTCCCCTGAAACGCACGCAGACGCAGCTGAGCCTGAATTCTTATCCACCCACGGCG
CCACCCTCCACTCCCGGAACGTAAGCCAAAAGATCAGAGAAAGAGACAGACAGAC
AAGATTAAAAGAGTGACAAGGCAAAGAGAGAGAAAAAAGAGTAAGAAACCGCCCT
AGAATCTTTTCAGTTTTTCCCTGTCTTCATGTGTCTTTGTTATAGAGACGCTGCCTTGT
CGGGGCAGCAGACTTGGGACGAGAGAGAGGCCTAGAGGGGGAGGAGTTGGTGAGC
GAGCTCGGGATCAGCGGGTGACCCCAGAGGTTGTTGCCTCTGGAAGTTCCCAATTCT
TTAACCCCCATCTTCTTCCACATGACTTTGGACTCGCTGCTGTTCTTTTGGTTGGTAT
ATCCTTGGATCTCTGACTCCGCCTGTTCCCCGAAATTTCCAGTTCTTGCTTCCTCTTG
GAGGAGGGCTGCAGCCTGCACCCTGCAGGGGAGAGAGAGCAGGAGGGGACGGAGA
CATGGGTAGCTTCGCTAAGCTGGCGAAGAGGGCGGTGGAGACGGACGCGCCGGTCA
TGGTGAAGGTGACGAATTTCTCCTACCTCGTCGAAATCGCATCTTGACTCTGGAGTT
TCTTGGGCTTCCTTGGTGAGTTCTTTCTAATAAGACGTCCCTTTTTCAGATACAAGAA
CTGCTTCGAGGGGCCACGGATGTCATGTCGCTTGCGCAGGTATATGACCATAATTTT
CTCGATTTGGCTGTGCAATGGTTTCCTGCTTTGTGATGTTATTTAAGGTTAATGGAAC
AAGCACGAAACGGAAAGTCAAACGAAAGCAGAACGGATTAATGGGAGTCGATCAT
CTTATAGCAATTCGCTCCCAAATGGTTTCTGTCAATTGTTCAAGTTGAAATGTACTAC
TGTAGTTCGGTACAGTTCATTTGCTACGTTTCGTGTAACAAATCATACTGTTTTTACT
TGCTATTCCTGAATAGTTCTTATGTTGCCGAAGTGGGACATTCGTATGTAATCTAACT
AGTGACATGCGTGTCTTTTAGTGTCCTATTAGCATGACCCGACCATATGATATTTATT
TCTGAGAAATTCGTTTCTGTTCTTCACTTTTTGTAAATCCTGAATGCAGGGAGTTGTT
TACTGGCAACCTCCTGAGTCAGCTCTGGATAAGATCGAAAAAATCGTCAGGGAACC
AACAGTCAGTAAGTATGGTTCTGATGATGGACTTCCTGAGCTTCGAGAAGCACTTCT
CGAAAAGGTAACTTCTTTTTATCATAGAATATATAATATATTTTGCCCATACACTGAC
TAAAGCAGGCCATGCTCTTGAGTATTACCACATTGCCATGCCTTTGTCATTGGGAAC
TAGGGATCCTGCTTCTAAACCACTACACCGTAACCAAATTTTAATAGAGATATTTGA
TCTTAACTCACAGTCGCATCAGCAATCTCAAGTGACCGATGCAATGGTTCAGAAATT
ACTTTGTCAAAACAAAGGTTCCAAAAAATCTTTTTGTTCCCAGAGTTACATTCCTGG
GAAGCATCTAATTTTCTGAATATTGGCAGAGTGTGTCATAAAACTCTCTATTCATCA
ACTGAGAAATGGGAACAATATTCATTATTGTGAGTTTGTGACTCCTCTGGAAATTCA
GAAGAGAAATTCATGGTGGTGCTAAGAGTTGCAAAGTCTCTCTGCCATGTTGTTTGT
CTAACAGTTGAGATGAAATCCTGAGATGATTTAGGTGCTCTAACCGTTAGCCTAAAT
GCTCCACATCGCACAGTTATCTTTAGAATTTGTTCCACCGTTTTGTCACCTACTGGGC
TAAGATTTTTATTGCTGTTTGGACCACCAATGTGTTAAAATTAGTACTGCATTGCAAG
CATCTTGTATATCTTTCCTGATGTCTACTGTATTTTTGGCAGCTACGCAGAGAGAATA
AGCTTACCAAGTCATCAGTCATGGTCACTGCTGGTGCAAATCAGGTAAAAACTTACT
TTCCTATTTTGCCTTGTAAAGTTTTCAAACTGGGCCTTACGTTTCCCAATTTACTTATG
TTTATTACTTCTTTAGGGGCACACTCTTCCTTTCCTTTTTCACTGCTAGACCTTGAACT
TGTCTTACAATCTGGTCTTTTGTTACATTGATTTTTTCAGGCTTTTGTGAACTTGGTCC
TCACTCTTTGTGATGCTGGTGATTCTGTTGTCATGTTTGCACCATATTATTTCAATGC
CTACATGTCATTCCAGATGACAGGTGTTACTGACATATTAATTGGTGGTTGCGATCC
CAAGACACTTCATCCTGATGTTGGTAAGATGATTCTCTTCTTCTTTCTGTTTCATTGG
AGAAATTGGTGAGATGTCTTATATCAATAGTCTGTTGTAAGAAATTTGTTGTACTTA
GTGCTGGTTCAGGAGCATCTGCTTCATTGGCACATGTTCAATTTCCGTTATAAGTCTA
CATTATCTTTTAGCTGATGAGAAGATATGCTTTTGTAAAATACAACGCATCTAGTAT
AAGAAATTATGCATTGGACGTAGTTGATAATATTTCATTTTGGAAGCATACATTACA
ATTATGTCTAAAACTGCAGATTGGTTGGAGAAGGTTCTGAAAGAAAATGACCCTATC
CCTAAACTTGTTACTGTTGTGAATCCGGGGAACCCCTCTGGAGCTTTTGTTCCCAGGC
CTATGCTTGAGGTCATCTCTCTCCACTAACACTTGTGTTTGGAATCACTATCCAATAT
TTCCTAATATTGCATTGTCATTCCAGAGAATTTCAGATCTGTGCAAAAATGCTGGTG
CATGGCTTGTGGTTGACAATACCTATGAGTAAGTCAATACTGTAAAGTGATCATTTT
CACAAGCAATTATATAATCTTTTGTGGTATCTGTTAGAGAGAACTTAGAAGAGCCGG
GGAATTATGGTTGTAGTCAAAGGGACAGATCATATAAGCCATTAGAGCTCTTCTGCC
TCCTCTTGAATCTGTGTCCAGATGAAAGACACTCTGATGGCATATAGGATTTCTAGT
ATCTTTGCAGTTTCCACAAGGAAGGGAAAATCAGAATATTGTGCCCATAGGAGATTC
AGAAAGTAGTCGTCCTGCACAGATATGTTGTGTGAGAATATGTCGAATACAGTTATC
CTGTGTTTGTTTTTTTGGTTTAGGATAAGTTCTCTTATTCAAATTCATTCCAGCTGCCA
AGCCTTTATAAATACGTTTTAGGCTCTAAACTTGGTGATGTTAAAAATAAGCCCTCC
AATCACACTTACTGCTTTATACTTTATACAGGCCAGGGTTTATGTTTGCCTGAAAGA
GTCAGGTGTTCAGGTCCATGTTGAGGAACTAATAGTAATTAATAAAGGACCTTTTCC
CTCTAAGCTTAAAGAAACATACAAGTTTTTGTCGTGGAGCTGCGATTAGCCAACCAT
ATTTGAAGTGGTAGCATAAAGAATGGAGTCCAAACATGTCGAGTGGAACACAGAAA
GTGGCTTATTCCTTTTACTCCCTGCAAACGAATTCTGCTCCTTATTGGAATTTAGTTA
TGGTCTTTAGCATTTGAGCTTTGTAAGATTTGATGCTTAGGTCAGTTTTCCTGTGTCT
TTTTGCAGATACTTTATGTATGATGGAATGGAGCACTATTGCTTAGAAGATACTCAT
ATTGTCAACCTCTTCTCATTCTCAAAGGCTTATGGAATGATGGGGTGGCGTGTAGGA
TACGTGAGTGCATCATACTCTTCGTTTATCATTTTATTATGCTTTACTCCACTTTTTGG
CTGCTGCTGCTGTTAATTGTCACTGTAACAGTAAGTCATACATGGACTGTCTGCTGC
AGATTGCATTTCCAAATGAAGCTGATGGCTTCCACGATCAGCTCCTCAAAGTGCAAG
ACAACATACCTATCTGTGCCTCCATCATCGGGCAGCGCCTGGCGCTCTACTCACTGG
AGGCTGGCCCCGAGTGGATCAAAGAAAGGGTGAAAGACTTGGTGAAAAACCGAGC
ACTGCTCGTGGAGGCGCTGTCCCCGCTCGGTGAGGACAATGTGAAGGGCGGGGAGG
GCGCCATCTACCTCTGGGCCAAACTGCCGGACAACTGCTCCAACGATTTTGAAGTTG
TCAGGTGGCTTGCAAACAAGCACGGTGTCGCTGTGATCCCTGGCAGCGCCAGTGGA
GGCCCCGGATACATCCGCGTCTCCTTCGGAGGGCTCAAAGAAGAAGACACCAGGCT
CGCTGCTGAGAGGCTAAGGCGCGGCTTGCAGGAGCTGGTGACTGATGGAATGGTAC
AGTAA
(Protein sequence in mutated form, comprising a tryptophan to
serine substitution at position 445
SEQ ID NO: 3
MVHGASATTQFMRAATQQQQQGKDRVPVGAGRQKPPRPRRGIGSSRTAACTTAERQA
ARPEPDASCPGAPAVRPRASALGRDPNPGRLSCLLWLSEQFLLPLGGGLQPAPCRGERA
GGDGDMGSFAKLAKRAVETDAPVMVKIQELLRGATDVMSLAQGVVYWQPPESALDKI
EKIVREPTVSKYGSDDGLPELREALLEKLRRENKLTKSSVMVTAGANQAFVNLVLTLCD
AGDSVVMFAPYYFNAYMSFQMTGVTDILIGGCDPKTLHPDVDWLEKVLKENDPIPKLV
TVVNPGNPSGAFVPRPMLERISDLCKNAGAWLVVDNTYEYFMYDGMEHYCLEDTHIV
NLFSFSKAYGMMGWRVGYIAFPNEADGFHDQLLKVQDNIPICASIIGQRLALYSLEAGPE
WIKERVKDLVKNRALLVEALSPLGEDNVKGGEGAIYLSAKLPDNCSNDFEVVRWLANK
HGVAVIPGSASGGPGYIRVSFGGLKEEDTRLAAERLRRGLQELVTDGMVQ
(protein sequence in wild type form comprising a tryptophan
amino acid at position 445
SEQ ID NO: 4
MVHGASATTQFMRAATQQQQQGKDRVPVGAGRQKPPRPRRGIGSSRTAACTTAERQA
ARPEPDASCPGAPAVRPRASALGRDPNPGRLSCLLWLSEQFLLPLGGGLQPAPCRGERA
GGDGDMGSFAKLAKRAVETDAPVMVKIQELLRGATDVMSLAQGVVYWQPPESALDKI
EKIVREPTVSKYGSDDGLPELREALLEKLRRENKLTKSSVMVTAGANQAFVNLVLTLCD
AGDSVVMFAPYYFNAYMSFQMTGVTDILIGGCDPKTLHPDVDWLEKVLKENDPIPKLV
TVVNPGNPSGAFVPRPMLERISDLCKNAGAWLVVDNTYEYFMYDGMEHYCLEDTHIV
NLFSFSKAYGMMGWRVGYIAFPNEADGFHDQLLKVQDNIPICASIIGQRLALYSLEAGPE
WIKERVKDLVKNRALLVEALSPLGEDNVKGGEGAIYLWAKLPDNCSNDFEVVRWLAN
KHGVAVIPGSASGGPGYIRVSFGGLKEEDTRLAAERLRRGLQELVTDGMVQ
(shows a preferred region for a primer or probe for detection of
the mutation of the invention)
SEQ ID NO: 11
CCCATAGGAGATTCAGAAAGTAGTCGTCCTGCACAGATATGTTGTGTGAGAATATGT
CGAATACAGTTATCCTGTGTTTGTTTTTTTGGTTTAGGATAAGTTCTCTTATTCAAATT
CATTCCAGCTGCCAAGCCTTTATAAATACGTTTTAGGCTCTAAACTTGGTGATGTTAA
AAATAAGCCCTCCAATCACACTTACTGCTTTATACTTTATACAGGCCAGGGTTTATGT
TTGCCTGAAAGAGTCAGGTGTTCAGGTCCATGTTGAGGAACTAATAGTAATTAATAA
AGGACCTTTTCCCTCTAAGCTTAAAGAAACATACAAGTTTTTGTCGTGGAGCTGCGA
TTAGCCAACCATATTTGAAGTGGTAGCATAAAGAATGGAGTCCAAACATGTCGAGT
GGAACACAGAAAGTGGCTTATTCCTTTTACTCCCTGCAAACGAATTCTGCTCCTTATT
GGAATTTAGTTATGGTCTTTAGCATTTGAGCTTTGTAAGATTTGATGCTTAGGTCAGT
TTTCCTGTGTCTTTTTGCAGATACTTTATGTATGATGGAATGGAGCACTATTGCTTAG
AAGATACTCATATTGTCAACCTCTTCTCATTCTCAAAGGCTTATGGAATGATGGGGT
GGCGTGTAGGATACGTGAGTGCATCATACTCTTCGTTTATCATTTTATTATGCTTTAC
TCCACTTTTTGGCTGCTGCTGCTGTTAATTGTCACTGTAACAGTAAGTCATACATGGA
CTGTCTGCTGCAGATTGCATTTCCAAATGAAGCTGATGGCTTCCACGATCAGCTCCT
CAAAGTGCAAGACAACATACCTATCTGTGCCTCCATCATCGGGCAGCGCCTGGCGCT
CTACTCACTGGAGGCTGGCCCCGAGTGGATCAAAGAAAGGGTGAAAGACTTGGTGA
AAAACCGAGCACTGCTCGTGGAGGCGCTGTCCCCGCTCGGTGAGGACAATGTGAAG
GGCGGGGAGGGCGCCATCTACCTCTSGGCCAAACTGCCGGACAACTGCTCCAACGA
TTTTGAAGTTGTCAGGTGGCTTGCAAACAAGCACGGTGTCGCTGTGATCCCTGGCAG
CGCCAGTGGAGGCCCCGGATACATCCGCGTCTCCTTCGGAGGGCTCAAAGAAGAAG
ACACCAGGCTCGCTGCTGAGAGGCTAAGGCGCGGCTTGCAGGAGCTGGTGACTGAT
GGAATGGTACAGTAACTGGTCCCTTGGTGTAAGTAAAAACGTAGCATTGAAGTTTTA
CAGAACTCCAATTTTCCAATGGTATAATTCTATACCGGGATTAATTCGAGAGAAGAC
TATTTTCAAGAATACATTGTTCGGACGAGAGCCGGGGACAGAATAAATAAAAGGTA
TCCGGTCTGCAGAGTAACCTATGTTATAAAATATAAATAGTACATATATCATCAGTG
AGTACTTGTTTCTGTTTGACATTCAGTTATCTATATTACTGATGGCAGCAATTGTACA
CAGTCAAAAGTATAAGCAAGAATTGTGAGCTGCTCTTGACGTACAGAGCTGCCTGTT
AGTAATTTGCTTGGCTTAGCTTGAGAATGAATACTAATGTCCAAATAACGTCAGCAT
CTGCCTGGATAATTCATAATTCAAGTGTCTGCAAGTAAGATAGATCTACTATATCTA
TAAAGCAGTGTTAAAAAGGACTCCTCAAGTTTGCACATAGAAATTACGGGATTAAT
AAAAAATGCAGTTGGATTTTAAGATAATCTAATGGTATAGATGTACTAGAAATTACC
AGAAAATAGAACCAATGGTTTTTTTTAATGACCTAATAGTCTAAATCCAAAGGAAAA
TGTATGCCAAATATAATCTGCATTTAAACTTTTTCCGTACCTACGGTGCTGTAAGCCA
AAAACTCTTCTCCTATTTTAATAAACACGTCTTCAAGGAAAAATATAAAAGTCATCA
AGACCAATAAAGTTTATTTATAGAAAAGTTACAGACATCAAATTACGGTTGGAAGA
ATAG
wherein S is C or G.
(Sobic.003G045600 [coding sequence only] Non-mutated codon)
SEQ ID. NO 12
ATGGTTCACGGCGCCTCCGCGACAACGCAATTCATGCGCGCCGCGACGCAGCAGCA
GCAGCAAGGAAAGGACCGCGTGCCCGTGGGCGCCGGTCGACAAAAACCTCCTCGCC
CCCGGAGGGGCATCGGATCCAGCCGAACCGCAGCGTGTACGACCGCGGAGCGGCAA
GCGGCGCGACCCGAACCCGACGCGAGCTGCCCGGGCGCACCGGCAGTCCGGCCTCG
TGCTTCTGCATTGGGTCGCGATCCAAATCCTGGGAGGCTCTCCTGTCTTCTCTGGCTA
TCCGAACAGTTCTTGCTTCCTCTTGGAGGAGGGCTGCAGCCTGCACCCTGCAGGGGA
GAGAGAGCAGGAGGGGACGGAGACATGGGTAGCTTCGCTAAGCTGGCGAAGAGGG
CGGTGGAGACGGACGCGCCGGTCATGGTGAAGATACAAGAACTGCTTCGAGGGGCC
ACGGATGTCATGTCGCTTGCGCAGGGAGTTGTTTACTGGCAACCTCCTGAGTCAGCT
CTGGATAAGATCGAAAAAATCGTCAGGGAACCAACAGTCAGTAAGTATGGTTCTGA
TGATGGACTTCCTGAGCTTCGAGAAGCACTTCTCGAAAAGCTACGCAGAGAGAATA
AGCTTACCAAGTCATCAGTCATGGTCACTGCTGGTGCAAATCAGGCTTTTGTGAACT
TGGTCCTCACTCTTTGTGATGCTGGTGATTCTGTTGTCATGTTTGCACCATATTATTTC
AATGCCTACATGTCATTCCAGATGACAGGTGTTACTGACATATTAATTGGTGGTTGC
GATCCCAAGACACTTCATCCTGATGTTGATTGGTTGGAGAAGGTTCTGAAAGAAAAT
GACCCTATCCCTAAACTTGTTACTGTTGTGAATCCGGGGAACCCCTCTGGAGCTTTTG
TTCCCAGGCCTATGCTTGAGAGAATTTCAGATCTGTGCAAAAATGCTGGTGCATGGC
TTGTGGTTGACAATACCTATGAATACTTTATGTATGATGGAATGGAGCACTATTGCT
TAGAAGATACTCATATTGTCAACCTCTTCTCATTCTCAAAGGCTTATGGAATGATGG
GGTGGCGTGTAGGATACATTGCATTTCCAAATGAAGCTGATGGCTTCCACGATCAGC
TCCTCAAAGTGCAAGACAACATACCTATCTGTGCCTCCATCATCGGGCAGCGCCTGG
CGCTCTACTCACTGGAGGCTGGCCCCGAGTGGATCAAAGAAAGGGTGAAAGACTTG
GTGAAAAACCGAGCACTGCTCGTGGAGGCGCTGTCCCCGCTCGGTGAGGACAATGT
GAAGGGCGGGGAGGGCGCCATCTACCTCTGGGCCAAACTGCCGGACAACTGCTCCA
ACGATTTTGAAGTTGTCAGGTGGCTTGCAAACAAGCACGGTGTCGCTGTGATCCCTG
GCAGCGCCAGTGGAGGCCCCGGATACATCCGCGTCTCCTTCGGAGGGCTCAAAGAA
GAAGACACCAGGCTCGCTGCTGAGAGGCTAAGGCGCGGCTTGCAGGAGCTGGTGAC
TGATGGAATGGTACAGTAA
(>Sobic.003G045600 [coding sequence only] Mutated codon in
ADV-HT2) Alternate Codon
SEQ ID NO: 13
ATGGTTCACGGCGCCTCCGCGACAACGCAATTCATGCGCGCCGCGACGCAGCAGCA
GCAGCAAGGAAAGGACCGCGTGCCCGTGGGCGCCGGTCGACAAAAACCTCCTCGCC
CCCGGAGGGGCATCGGATCCAGCCGAACCGCAGCGTGTACGACCGCGGAGCGGCAA
GCGGCGCGACCCGAACCCGACGCGAGCTGCCCGGGCGCACCGGCAGTCCGGCCTCG
TGCTTCTGCATTGGGTCGCGATCCAAATCCTGGGAGGCTCTCCTGTCTTCTCTGGCTA
TCCGAACAGTTCTTGCTTCCTCTTGGAGGAGGGCTGCAGCCTGCACCCTGCAGGGGA
GAGAGAGCAGGAGGGGACGGAGACATGGGTAGCTTCGCTAAGCTGGCGAAGAGGG
CGGTGGAGACGGACGCGCCGGTCATGGTGAAGATACAAGAACTGCTTCGAGGGGCC
ACGGATGTCATGTCGCTTGCGCAGGGAGTTGTTTACTGGCAACCTCCTGAGTCAGCT
CTGGATAAGATCGAAAAAATCGTCAGGGAACCAACAGTCAGTAAGTATGGTTCTGA
TGATGGACTTCCTGAGCTTCGAGAAGCACTTCTCGAAAAGCTACGCAGAGAGAATA
AGCTTACCAAGTCATCAGTCATGGTCACTGCTGGTGCAAATCAGGCTTTTGTGAACT
TGGTCCTCACTCTTTGTGATGCTGGTGATTCTGTTGTCATGTTTGCACCATATTATTTC
AATGCCTACATGTCATTCCAGATGACAGGTGTTACTGACATATTAATTGGTGGTTGC
GATCCCAAGACACTTCATCCTGATGTTGATTGGTTGGAGAAGGTTCTGAAAGAAAAT
GACCCTATCCCTAAACTTGTTACTGTTGTGAATCCGGGGAACCCCTCTGGAGCTTTTG
TTCCCAGGCCTATGCTTGAGAGAATTTCAGATCTGTGCAAAAATGCTGGTGCATGGC
TTGTGGTTGACAATACCTATGAATACTTTATGTATGATGGAATGGAGCACTATTGCT
TAGAAGATACTCATATTGTCAACCTCTTCTCATTCTCAAAGGCTTATGGAATGATGG
GGTGGCGTGTAGGATACATTGCATTTCCAAATGAAGCTGATGGCTTCCACGATCAGC
TCCTCAAAGTGCAAGACAACATACCTATCTGTGCCTCCATCATCGGGCAGCGCCTGG
CGCTCTACTCACTGGAGGCTGGCCCCGAGTGGATCAAAGAAAGGGTGAAAGACTTG
GTGAAAAACCGAGCACTGCTCGTGGAGGCGCTGTCCCCGCTCGGTGAGGACAATGT
GAAGGGCGGGGAGGGCGCCATCTACCTCTCGGCCAAACTGCCGGACAACTGCTCCA
ACGATTTTGAAGTTGTCAGGTGGCTTGCAAACAAGCACGGTGTCGCTGTGATCCCTG
GCAGCGCCAGTGGAGGCCCCGGATACATCCGCGTCTCCTTCGGAGGGCTCAAAGAA
GAAGACACCAGGCTCGCTGCTGAGAGGCTAAGGCGCGGCTTGCAGGAGCTGGTGAC
TGATGGAATGGTACAGTAA
(>Sobic.003G045600 [coding sequence only] Alternative
mutated codon 1) Alternate Codon
SEQ ID NO: 14
ATGGTTCACGGCGCCTCCGCGACAACGCAATTCATGCGCGCCGCGACGCAGCAGCA
GCAGCAAGGAAAGGACCGCGTGCCCGTGGGCGCCGGTCGACAAAAACCTCCTCGCC
CCCGGAGGGGCATCGGATCCAGCCGAACCGCAGCGTGTACGACCGCGGAGCGGCAA
GCGGCGCGACCCGAACCCGACGCGAGCTGCCCGGGCGCACCGGCAGTCCGGCCTCG
TGCTTCTGCATTGGGTCGCGATCCAAATCCTGGGAGGCTCTCCTGTCTTCTCTGGCTA
TCCGAACAGTTCTTGCTTCCTCTTGGAGGAGGGCTGCAGCCTGCACCCTGCAGGGGA
GAGAGAGCAGGAGGGGACGGAGACATGGGTAGCTTCGCTAAGCTGGCGAAGAGGG
CGGTGGAGACGGACGCGCCGGTCATGGTGAAGATACAAGAACTGCTTCGAGGGGCC
ACGGATGTCATGTCGCTTGCGCAGGGAGTTGTTTACTGGCAACCTCCTGAGTCAGCT
CTGGATAAGATCGAAAAAATCGTCAGGGAACCAACAGTCAGTAAGTATGGTTCTGA
TGATGGACTTCCTGAGCTTCGAGAAGCACTTCTCGAAAAGCTACGCAGAGAGAATA
AGCTTACCAAGTCATCAGTCATGGTCACTGCTGGTGCAAATCAGGCTTTTGTGAACT
TGGTCCTCACTCTTTGTGATGCTGGTGATTCTGTTGTCATGTTTGCACCATATTATTTC
AATGCCTACATGTCATTCCAGATGACAGGTGTTACTGACATATTAATTGGTGGTTGC
GATCCCAAGACACTTCATCCTGATGTTGATTGGTTGGAGAAGGTTCTGAAAGAAAAT
GACCCTATCCCTAAACTTGTTACTGTTGTGAATCCGGGGAACCCCTCTGGAGCTTTTG
TTCCCAGGCCTATGCTTGAGAGAATTTCAGATCTGTGCAAAAATGCTGGTGCATGGC
TTGTGGTTGACAATACCTATGAATACTTTATGTATGATGGAATGGAGCACTATTGCT
TAGAAGATACTCATATTGTCAACCTCTTCTCATTCTCAAAGGCTTATGGAATGATGG
GGTGGCGTGTAGGATACATTGCATTTCCAAATGAAGCTGATGGCTTCCACGATCAGC
TCCTCAAAGTGCAAGACAACATACCTATCTGTGCCTCCATCATCGGGCAGCGCCTGG
CGCTCTACTCACTGGAGGCTGGCCCCGAGTGGATCAAAGAAAGGGTGAAAGACTTG
GTGAAAAACCGAGCACTGCTCGTGGAGGCGCTGTCCCCGCTCGGTGAGGACAATGT
GAAGGGCGGGGAGGGCGCCATCTACCTCTCTGCCAAACTGCCGGACAACTGCTCCA
ACGATTTTGAAGTTGTCAGGTGGCTTGCAAACAAGCACGGTGTCGCTGTGATCCCTG
GCAGCGCCAGTGGAGGCCCCGGATACATCCGCGTCTCCTTCGGAGGGCTCAAAGAA
GAAGACACCAGGCTCGCTGCTGAGAGGCTAAGGCGCGGCTTGCAGGAGCTGGTGAC
TGATGGAATGGTACAGTAA
(Sobic.003G045600 [coding sequence only] Alternative mutated
codon 2) Alternate Codon
SEQ ID NO: 15
ATGGTTCACGGCGCCTCCGCGACAACGCAATTCATGCGCGCCGCGACGCAGCAGCA
GCAGCAAGGAAAGGACCGCGTGCCCGTGGGCGCCGGTCGACAAAAACCTCCTCGCC
CCCGGAGGGGCATCGGATCCAGCCGAACCGCAGCGTGTACGACCGCGGAGCGGCAA
GCGGCGCGACCCGAACCCGACGCGAGCTGCCCGGGCGCACCGGCAGTCCGGCCTCG
TGCTTCTGCATTGGGTCGCGATCCAAATCCTGGGAGGCTCTCCTGTCTTCTCTGGCTA
TCCGAACAGTTCTTGCTTCCTCTTGGAGGAGGGCTGCAGCCTGCACCCTGCAGGGGA
GAGAGAGCAGGAGGGGACGGAGACATGGGTAGCTTCGCTAAGCTGGCGAAGAGGG
CGGTGGAGACGGACGCGCCGGTCATGGTGAAGATACAAGAACTGCTTCGAGGGGCC
ACGGATGTCATGTCGCTTGCGCAGGGAGTTGTTTACTGGCAACCTCCTGAGTCAGCT
CTGGATAAGATCGAAAAAATCGTCAGGGAACCAACAGTCAGTAAGTATGGTTCTGA
TGATGGACTTCCTGAGCTTCGAGAAGCACTTCTCGAAAAGCTACGCAGAGAGAATA
AGCTTACCAAGTCATCAGTCATGGTCACTGCTGGTGCAAATCAGGCTTTTGTGAACT
TGGTCCTCACTCTTTGTGATGCTGGTGATTCTGTTGTCATGTTTGCACCATATTATTTC
AATGCCTACATGTCATTCCAGATGACAGGTGTTACTGACATATTAATTGGTGGTTGC
GATCCCAAGACACTTCATCCTGATGTTGATTGGTTGGAGAAGGTTCTGAAAGAAAAT
GACCCTATCCCTAAACTTGTTACTGTTGTGAATCCGGGGAACCCCTCTGGAGCTTTTG
TTCCCAGGCCTATGCTTGAGAGAATTTCAGATCTGTGCAAAAATGCTGGTGCATGGC
TTGTGGTTGACAATACCTATGAATACTTTATGTATGATGGAATGGAGCACTATTGCT
TAGAAGATACTCATATTGTCAACCTCTTCTCATTCTCAAAGGCTTATGGAATGATGG
GGTGGCGTGTAGGATACATTGCATTTCCAAATGAAGCTGATGGCTTCCACGATCAGC
TCCTCAAAGTGCAAGACAACATACCTATCTGTGCCTCCATCATCGGGCAGCGCCTGG
CGCTCTACTCACTGGAGGCTGGCCCCGAGTGGATCAAAGAAAGGGTGAAAGACTTG
GTGAAAAACCGAGCACTGCTCGTGGAGGCGCTGTCCCCGCTCGGTGAGGACAATGT
GAAGGGCGGGGAGGGCGCCATCTACCTCTCCGCCAAACTGCCGGACAACTGCTCCA
ACGATTTTGAAGTTGTCAGGTGGCTTGCAAACAAGCACGGTGTCGCTGTGATCCCTG
GCAGCGCCAGTGGAGGCCCCGGATACATCCGCGTCTCCTTCGGAGGGCTCAAAGAA
GAAGACACCAGGCTCGCTGCTGAGAGGCTAAGGCGCGGCTTGCAGGAGCTGGTGAC
TGATGGAATGGTACAGTAA
(>Sobic.003G045600 [coding sequence only] Alternative
mutated codon 3) Alternate Codon
SEQ ID NO: 16
ATGGTTCACGGCGCCTCCGCGACAACGCAATTCATGCGCGCCGCGACGCAGCAGCA
GCAGCAAGGAAAGGACCGCGTGCCCGTGGGCGCCGGTCGACAAAAACCTCCTCGCC
CCCGGAGGGGCATCGGATCCAGCCGAACCGCAGCGTGTACGACCGCGGAGCGGCAA
GCGGCGCGACCCGAACCCGACGCGAGCTGCCCGGGCGCACCGGCAGTCCGGCCTCG
TGCTTCTGCATTGGGTCGCGATCCAAATCCTGGGAGGCTCTCCTGTCTTCTCTGGCTA
TCCGAACAGTTCTTGCTTCCTCTTGGAGGAGGGCTGCAGCCTGCACCCTGCAGGGGA
GAGAGAGCAGGAGGGGACGGAGACATGGGTAGCTTCGCTAAGCTGGCGAAGAGGG
CGGTGGAGACGGACGCGCCGGTCATGGTGAAGATACAAGAACTGCTTCGAGGGGCC
ACGGATGTCATGTCGCTTGCGCAGGGAGTTGTTTACTGGCAACCTCCTGAGTCAGCT
CTGGATAAGATCGAAAAAATCGTCAGGGAACCAACAGTCAGTAAGTATGGTTCTGA
TGATGGACTTCCTGAGCTTCGAGAAGCACTTCTCGAAAAGCTACGCAGAGAGAATA
AGCTTACCAAGTCATCAGTCATGGTCACTGCTGGTGCAAATCAGGCTTTTGTGAACT
TGGTCCTCACTCTTTGTGATGCTGGTGATTCTGTTGTCATGTTTGCACCATATTATTTC
AATGCCTACATGTCATTCCAGATGACAGGTGTTACTGACATATTAATTGGTGGTTGC
GATCCCAAGACACTTCATCCTGATGTTGATTGGTTGGAGAAGGTTCTGAAAGAAAAT
GACCCTATCCCTAAACTTGTTACTGTTGTGAATCCGGGGAACCCCTCTGGAGCTTTTG
TTCCCAGGCCTATGCTTGAGAGAATTTCAGATCTGTGCAAAAATGCTGGTGCATGGC
TTGTGGTTGACAATACCTATGAATACTTTATGTATGATGGAATGGAGCACTATTGCT
TAGAAGATACTCATATTGTCAACCTCTTCTCATTCTCAAAGGCTTATGGAATGATGG
GGTGGCGTGTAGGATACATTGCATTTCCAAATGAAGCTGATGGCTTCCACGATCAGC
TCCTCAAAGTGCAAGACAACATACCTATCTGTGCCTCCATCATCGGGCAGCGCCTGG
CGCTCTACTCACTGGAGGCTGGCCCCGAGTGGATCAAAGAAAGGGTGAAAGACTTG
GTGAAAAACCGAGCACTGCTCGTGGAGGCGCTGTCCCCGCTCGGTGAGGACAATGT
GAAGGGCGGGGAGGGCGCCATCTACCTCTCAGCCAAACTGCCGGACAACTGCTCCA
ACGATTTTGAAGTTGTCAGGTGGCTTGCAAACAAGCACGGTGTCGCTGTGATCCCTG
GCAGCGCCAGTGGAGGCCCCGGATACATCCGCGTCTCCTTCGGAGGGCTCAAAGAA
GAAGACACCAGGCTCGCTGCTGAGAGGCTAAGGCGCGGCTTGCAGGAGCTGGTGAC
TGATGGAATGGTACAGTAA
(>Sobic.003G045600 [coding sequence only] Alternative
mutated codon 4) Alternate Codon
SEQ ID NO: 17
ATGGTTCACGGCGCCTCCGCGACAACGCAATTCATGCGCGCCGCGACGCAGCAGCA
GCAGCAAGGAAAGGACCGCGTGCCCGTGGGCGCCGGTCGACAAAAACCTCCTCGCC
CCCGGAGGGGCATCGGATCCAGCCGAACCGCAGCGTGTACGACCGCGGAGCGGCAA
GCGGCGCGACCCGAACCCGACGCGAGCTGCCCGGGCGCACCGGCAGTCCGGCCTCG
TGCTTCTGCATTGGGTCGCGATCCAAATCCTGGGAGGCTCTCCTGTCTTCTCTGGCTA
TCCGAACAGTTCTTGCTTCCTCTTGGAGGAGGGCTGCAGCCTGCACCCTGCAGGGGA
GAGAGAGCAGGAGGGGACGGAGACATGGGTAGCTTCGCTAAGCTGGCGAAGAGGG
CGGTGGAGACGGACGCGCCGGTCATGGTGAAGATACAAGAACTGCTTCGAGGGGCC
ACGGATGTCATGTCGCTTGCGCAGGGAGTTGTTTACTGGCAACCTCCTGAGTCAGCT
CTGGATAAGATCGAAAAAATCGTCAGGGAACCAACAGTCAGTAAGTATGGTTCTGA
TGATGGACTTCCTGAGCTTCGAGAAGCACTTCTCGAAAAGCTACGCAGAGAGAATA
AGCTTACCAAGTCATCAGTCATGGTCACTGCTGGTGCAAATCAGGCTTTTGTGAACT
TGGTCCTCACTCTTTGTGATGCTGGTGATTCTGTTGTCATGTTTGCACCATATTATTTC
AATGCCTACATGTCATTCCAGATGACAGGTGTTACTGACATATTAATTGGTGGTTGC
GATCCCAAGACACTTCATCCTGATGTTGATTGGTTGGAGAAGGTTCTGAAAGAAAAT
GACCCTATCCCTAAACTTGTTACTGTTGTGAATCCGGGGAACCCCTCTGGAGCTTTTG
TTCCCAGGCCTATGCTTGAGAGAATTTCAGATCTGTGCAAAAATGCTGGTGCATGGC
TTGTGGTTGACAATACCTATGAATACTTTATGTATGATGGAATGGAGCACTATTGCT
TAGAAGATACTCATATTGTCAACCTCTTCTCATTCTCAAAGGCTTATGGAATGATGG
GGTGGCGTGTAGGATACATTGCATTTCCAAATGAAGCTGATGGCTTCCACGATCAGC
TCCTCAAAGTGCAAGACAACATACCTATCTGTGCCTCCATCATCGGGCAGCGCCTGG
CGCTCTACTCACTGGAGGCTGGCCCCGAGTGGATCAAAGAAAGGGTGAAAGACTTG
GTGAAAAACCGAGCACTGCTCGTGGAGGCGCTGTCCCCGCTCGGTGAGGACAATGT
GAAGGGCGGGGAGGGCGCCATCTACCTCAGTGCCAAACTGCCGGACAACTGCTCCA
ACGATTTTGAAGTTGTCAGGTGGCTTGCAAACAAGCACGGTGTCGCTGTGATCCCTG
GCAGCGCCAGTGGAGGCCCCGGATACATCCGCGTCTCCTTCGGAGGGCTCAAAGAA
GAAGACACCAGGCTCGCTGCTGAGAGGCTAAGGCGCGGCTTGCAGGAGCTGGTGAC
TGATGGAATGGTACAGTAA
(>Sobic.003G045600 [coding sequence only] Alternative
mutated codon 5] Alternate Codon
SEQ ID NO: 18
ATGGTTCACGGCGCCTCCGCGACAACGCAATTCATGCGCGCCGCGACGCAGCAGCA
GCAGCAAGGAAAGGACCGCGTGCCCGTGGGCGCCGGTCGACAAAAACCTCCTCGCC
CCCGGAGGGGCATCGGATCCAGCCGAACCGCAGCGTGTACGACCGCGGAGCGGCAA
GCGGCGCGACCCGAACCCGACGCGAGCTGCCCGGGCGCACCGGCAGTCCGGCCTCG
TGCTTCTGCATTGGGTCGCGATCCAAATCCTGGGAGGCTCTCCTGTCTTCTCTGGCTA
TCCGAACAGTTCTTGCTTCCTCTTGGAGGAGGGCTGCAGCCTGCACCCTGCAGGGGA
GAGAGAGCAGGAGGGGACGGAGACATGGGTAGCTTCGCTAAGCTGGCGAAGAGGG
CGGTGGAGACGGACGCGCCGGTCATGGTGAAGATACAAGAACTGCTTCGAGGGGCC
ACGGATGTCATGTCGCTTGCGCAGGGAGTTGTTTACTGGCAACCTCCTGAGTCAGCT
CTGGATAAGATCGAAAAAATCGTCAGGGAACCAACAGTCAGTAAGTATGGTTCTGA
TGATGGACTTCCTGAGCTTCGAGAAGCACTTCTCGAAAAGCTACGCAGAGAGAATA
AGCTTACCAAGTCATCAGTCATGGTCACTGCTGGTGCAAATCAGGCTTTTGTGAACT
TGGTCCTCACTCTTTGTGATGCTGGTGATTCTGTTGTCATGTTTGCACCATATTATTTC
AATGCCTACATGTCATTCCAGATGACAGGTGTTACTGACATATTAATTGGTGGTTGC
GATCCCAAGACACTTCATCCTGATGTTGATTGGTTGGAGAAGGTTCTGAAAGAAAAT
GACCCTATCCCTAAACTTGTTACTGTTGTGAATCCGGGGAACCCCTCTGGAGCTTTTG
TTCCCAGGCCTATGCTTGAGAGAATTTCAGATCTGTGCAAAAATGCTGGTGCATGGC
TTGTGGTTGACAATACCTATGAATACTTTATGTATGATGGAATGGAGCACTATTGCT
TAGAAGATACTCATATTGTCAACCTCTTCTCATTCTCAAAGGCTTATGGAATGATGG
GGTGGCGTGTAGGATACATTGCATTTCCAAATGAAGCTGATGGCTTCCACGATCAGC
TCCTCAAAGTGCAAGACAACATACCTATCTGTGCCTCCATCATCGGGCAGCGCCTGG
CGCTCTACTCACTGGAGGCTGGCCCCGAGTGGATCAAAGAAAGGGTGAAAGACTTG
GTGAAAAACCGAGCACTGCTCGTGGAGGCGCTGTCCCCGCTCGGTGAGGACAATGT
GAAGGGCGGGGAGGGCGCCATCTACCTCAGCGCCAAACTGCCGGACAACTGCTCCA
ACGATTTTGAAGTTGTCAGGTGGCTTGCAAACAAGCACGGTGTCGCTGTGATCCCTG
GCAGCGCCAGTGGAGGCCCCGGATACATCCGCGTCTCCTTCGGAGGGCTCAAAGAA
GAAGACACCAGGCTCGCTGCTGAGAGGCTAAGGCGCGGCTTGCAGGAGCTGGTGAC
TGATGGAATGGTACAGTAA

1. An amino acid sequence having SEQ ID NO: 3, comprising a mutation from a Tryptophane to a Serine residue at position 445.

2. A nucleic acid encoding the amino acid sequence of claim 1.

3. The nucleic acid of claim 2, comprising SEQ ID NO: 1.

4. A plant or seed comprising the nucleic acid of claim 2, which provides resistance or tolerance to an HPPD herbicide in the plant or seed.

5. The plant or seed of claim 4, wherein the plant or seed is a sorghum plant or seed.

6. The plant or seed of claim 4, which is present in a non-HPPD gene located on chromosome 3 of the plant.

7. The nucleic acid as claimed in claim 2, comprising any one of alternative codons TCT, TCC, TCA, AGT, AGC encoding the Serine residue at amino acid position 445 of SEQ ID NO: 3.

8. A method of determining the presence or absence of a mutation on chromosome 3 of a plant with HPPD herbicide tolerance or resistance comprising:
a) providing a nucleic acid sample from the plant;
b) amplifying a region comprising the mutation on chromosome 3 present in said nucleic acid sample from the plant using primers; and
c) identifying a herbicide-resistant plant based on the presence of at least one mutation on chromosome 3 in the amplified nucleic acid sample.

9. The method of claim 8, wherein the primers are primers of SEQ ID NOs: 1, 11, 13, 5, 6, 7, or 8.

10. A sorghum plant or seed, comprising a nucleic acid encoding a Serine residue at amino acid position 445 of SES ID NO:3.

11. The sorghum plant or seed of claim 10, which has enhanced resistance or tolerance to an HPPD herbicide.

12. A method of producing a sorghum plant or seed comprising introducing the mutation as defined in claim 1 into the sorghum plant or seed.

13. The method of claim 12, wherein the sorghum plant is a heterozygous or a homozygous plant.

14. The sorghum plant of claim 10, representative seeds of which having been deposited under accession number NCIMB 43919.

15. A method of controlling weeds in the vicinity of the sorghum plant of claim 10, comprising applying an effective amount of a HPPD inhibitor herbicide to a weed or the plant.

16. The nucleic acid as claimed in claim 4, wherein the HPPD inhibitor herbicide is selected from benzoylpyrazole herbicides, benzoylcyclohexanedione herbicides, aroylcyclohexanedione herbicides, oxazole herbicides, cyclopropylisoxazole herbicides, pyrazolone, carbobicyclic compounds, cyclic ketone compounds, benzoylpyrazole herbicides, pyrazole herbicides, triketone herbicides, aroylcyclohexanedione herbicides, and in particular from tolpyralate. fenquinotrione, mesotrione, tefuryltrione, isoxaflutole, pyrasulfotole, benzobicyclon, benzofenap, pyrazolynate, pyrazoxyfen, bicyclopyrone, sulcotrione, tembotrione, topramezone, and a combination thereof.

17. The method as claimed in claim 15, wherein the HPPD inhibitor herbicide is selected from benzoylpyrazole herbicides, benzoylcyclohexanedione herbicides, aroylcyclohexanedione herbicides, oxazole herbicides, cyclopropylisoxazole herbicides, pyrazolone, carbobicyclic compounds, cyclic ketone compounds, benzoylpyrazole herbicides, pyrazole herbicides, triketone herbicides, aroylcyclohexanedione herbicides, and in particular from tolpyralate. fenquinotrione, mesotrione, tefuryltrione, isoxaflutole, pyrasulfotole, benzobicyclon, benzofenap, pyrazolynate, pyrazoxyfen, bicyclopyrone, sulcotrione, tembotrione, topramezone, and a combination thereof.

18. The plant as claimed in claim 10, wherein the HPPD inhibitor herbicide is selected from benzoylpyrazole herbicides, benzoylcyclohexanedione herbicides, aroylcyclohexanedione herbicides, oxazole herbicides, cyclopropylisoxazole herbicides, pyrazolone, carbobicyclic compounds, cyclic ketone compounds, benzoylpyrazole herbicides, pyrazole herbicides, triketone herbicides, aroylcyclohexanedione herbicides, and in particular from tolpyralate. fenquinotrione, mesotrione, tefuryltrione, isoxaflutole, pyrasulfotole, benzobicyclon, benzofenap, pyrazolynate, pyrazoxyfen, bicyclopyrone, sulcotrione, tembotrione, topramezone, and a combination thereof.

19. The method of claim 15, comprising applying the herbicide at a concentration of 0.5×-5× of the recommended dose

20. The method of claim 15, comprising applying the herbicide at a concentration of 0.5×-4× of the recommended dose.


SEQUENCE LISTING (21 sequences)

SEQ ID NO: 1 - DNA, 5497 bp, Sorghum bicolor
  source 1..5497: mol_type = genomic DNA
atggttcacggcgcctccgcgacaacgcaattcatgcgcgccgcgacgcagcagcagcagcaaggaaaggaccgcgtgcccgtgggcgccggtcgacaaaaacctcctcgcccccggaggggcatcggatccagccgaaccgcagcgtgtacgaccgcggagcggcaagcggcgcgacccgaacccgacgcgagctgcccgggcgcaccggcagtccggcctcgtgcttctgcattgggtcgcgatccaaatcctgggaggctctcctgtcttctctggctatccgaacaggtccgtccgagcattatttgtcagatctctttccgtcttgtcagttgtccacggtctccgagccctcctatttgcatccacgtagattgatgatagattcctctcggaagtgtctcgaaattttctaaactctaaaattagaaagttagtacttgtgaaattctttaaaaaaaaaattactagtaaaactgagctgcgctaaacagatttcgccagatttgaagatctgagatttcgagattatctagtgcagagggacatttcttattttttaccttctcaaatttgaaacactaaacgacttcttttttcagcaggagagactcagattctgatcgcatttttttttcactgatcacatttgtgcttctctctcgtaggccttgtttagttccgaaaagtgaaaactttttggaactgtagcattttcgtttgtttgatcatggactaactaggatcaaaagattcgtcttgtgatttacagctaaactgtgtaattagtttttgttttcgtttatatttaatgtttcatgcatgtgacacaagattcgatgtgacgggaaatcttgaaaactttttggttttcagggtaaactaaacaaggccgtagtcgttctggacaagttttgtcctaacgagcagaagcgtactgacgaaacttgtcttcactggcccattggatcttgtggcagctcatgcaaaaaatatttcgatagttgcctatcacacgcatgtgtttactgtccacagacgctgattctaaagaatcttttcaccatcaattggccgcgacattttggcgatttctctgcgagactcactgatgacacattacttttgcatttattaaaaaatccaatctttttgtctctttttacccgaaaaaagctccctccttttttgacccctcttccttcccctgaaacgcacgcagacgcagctgagcctgaattcttatccacccacggcgccaccctccactcccggaacgtaagccaaaagatcagagaaagagacagacagacaagattaaaagagtgacaaggcaaagagagagaaaaaagagtaagaaaccgccctagaatcttttcagtttttccctgtcttcatgtgtctttgttatagagacgctgccttgtcggggcagcagacttgggacgagagagaggcctagagggggaggagttggtgagcgagctcgggatcagcgggtgaccccagaggttgttgcctctggaagttcccaattctttaacccccatcttcttccacatgactttggactcgctgctgttcttttggttggtatatccttggatctctgactccgcctgttccccgaaatttccagttcttgcttcctcttggaggagggctgcagcctgcaccctgcaggggagagagagcaggaggggacggagacatgggtagcttcgctaagctggcgaagagggcggtggagacggacgcgccggtcatggtgaaggtgacgaatttctcctacctcgtcgaaatcgcatcttgactctggagtttcttgggcttccttggtgagttctttctaataagacgtccctttttcagatacaagaactgcttcgaggggccacggatgtcatgtcgcttgcgcaggtatatgaccataattttctcgatttggctgtgcaatggtttcctgctttgtgatgttatttaaggttaatggaacaagcacgaaacggaaagtcaaacgaaagcagaacggattaatgggagtcgatcatcttatagcaattcgctcccaaatggtttctgtcaattgttcaagttgaaatgtactactgtagttcggtacagttcatttgctacgtttcgtgtaacaaatcatactgtttttacttgctattcctgaatagttcttatgttgccgaagtgggacattcgtatgtaatctaactagtgacatgcgtgtcttttagtgtcctattagcatgacccgaccatatgatatttatttctgagaaattcgtttctgttcttcactttttgtaaatcctgaatgcagggagttgtttactggcaacctcctgagtcagctctggataagatcgaaaaaatcgtcagggaaccaacagtcagtaagtatggttctgatgatggacttcctgagcttcgagaagcacttctcgaaaaggtaacttctttttatcatagaatatataatatattttgcccatacactgactaaagcaggccatgctcttgagtattaccacattgccatgcctttgtcattgggaactagggatcctgcttctaaaccactacaccgtaaccaaattttaatagagatatttgatcttaactcacagtcgcatcagcaatctcaagtgaccgatgcaatggttcagaaattactttgtcaaaacaaaggttccaaaaaatctttttgttcccagagttacattcctgggaagcatctaattttctgaatattggcagagtgtgtcataaaactctctattcatcaactgagaaatgggaacaatattcattattgtgagtttgtgactcctctggaaattcagaagagaaattcatggtggtgctaagagttgcaaagtctctctgccatgttgtttgtctaacagttgagatgaaatcctgagatgatttaggtgctctaaccgttagcctaaatgctccacatcgcacagttatctttagaatttgttccaccgttttgtcacctactgggctaagatttttattgctgtttggaccaccaatgtgttaaaattagtactgcattgcaagcatcttgtatatctttcctgatgtctactgtatttttggcagctacgcagagagaataagcttaccaagtcatcagtcatggtcactgctggtgcaaatcaggtaaaaacttactttcctattttgccttgtaaagttttcaaactgggccttacgtttcccaatttacttatgtttattacttctttaggggcacactcttcctttcctttttcactgctagaccttgaacttgtcttacaatctggtcttttgttacattgattttttcaggcttttgtgaacttggtcctcactctttgtgatgctggtgattctgttgtcatgtttgcaccatattatttcaatgcctacatgtcattccagatgacaggtgttactgacatattaattggtggttgcgatcccaagacacttcatcctgatgttggtaagatgattctcttcttctttctgtttcattggagaaattggtgagatgtcttatatcaatagtctgttgtaagaaatttgttgtacttagtgctggttcaggagcatctgcttcattggcacatgttcaatttccgttataagtctacattatcttttagctgatgagaagatatgcttttgtaaaatacaacgcatctagtataagaaattatgcattggacgtagttgataatatttcattttggaagcatacattacaattatgtctaaaactgcagattggttggagaaggttctgaaagaaaatgaccctatccctaaacttgttactgttgtgaatccggggaacccctctggagcttttgttcccaggcctatgcttgaggtcatctctctccactaacacttgtgtttggaatcactatccaatatttcctaatattgcattgtcattccagagaatttcagatctgtgcaaaaatgctggtgcatggcttgtggttgacaatacctatgagtaagtcaatactgtaaagtgatcattttcacaagcaattatataatcttttgtggtatctgttagagagaacttagaagagccggggaattatggttgtagtcaaagggacagatcatataagccattagagctcttctgcctcctcttgaatctgtgtccagatgaaagacactctgatggcatataggatttctagtatctttgcagtttccacaaggaagggaaaatcagaatattgtgcccataggagattcagaaagtagtcgtcctgcacagatatgttgtgtgagaatatgtcgaatacagttatcctgtgtttgtttttttggtttaggataagttctcttattcaaattcattccagctgccaagcctttataaatacgttttaggctctaaacttggtgatgttaaaaataagccctccaatcacacttactgctttatactttatacaggccagggtttatgtttgcctgaaagagtcaggtgttcaggtccatgttgaggaactaatagtaattaataaaggaccttttccctctaagcttaaagaaacatacaagtttttgtcgtggagctgcgattagccaaccatatttgaagtggtagcataaagaatggagtccaaacatgtcgagtggaacacagaaagtggcttattccttttactccctgcaaacgaattctgctccttattggaatttagttatggtctttagcatttgagctttgtaagatttgatgcttaggtcagttttcctgtgtctttttgcagatactttatgtatgatggaatggagcactattgcttagaagatactcatattgtcaacctcttctcattctcaaaggcttatggaatgatggggtggcgtgtaggatacgtgagtgcatcatactcttcgtttatcattttattatgctttactccactttttggctgctgctgctgttaattgtcactgtaacagtaagtcatacatggactgtctgctgcagattgcatttccaaatgaagctgatggcttccacgatcagctcctcaaagtgcaagacaacatacctatctgtgcctccatcatcgggcagcgcctggcgctctactcactggaggctggccccgagtggatcaaagaaagggtgaaagacttggtgaaaaaccgagcactgctcgtggaggcgctgtccccgctcggtgaggacaatgtgaagggcggggagggcgccatctacctctcggccaaactgccggacaactgctccaacgattttgaagttgtcaggtggcttgcaaacaagcacggtgtcgctgtgatccctggcagcgccagtggaggccccggatacatccgcgtctccttcggagggctcaaagaagaagacaccaggctcgctgctgagaggctaaggcgcggcttgcaggagctggtgactgatggaatggtacagtaa

SEQ ID NO: 2 - DNA, 5497 bp, Sorghum bicolor
  source 1..5497: mol_type = genomic DNA
atggttcacggcgcctccgcgacaacgcaattcatgcgcgccgcgacgcagcagcagcagcaaggaaaggaccgcgtgcccgtgggcgccggtcgacaaaaacctcctcgcccccggaggggcatcggatccagccgaaccgcagcgtgtacgaccgcggagcggcaagcggcgcgacccgaacccgacgcgagctgcccgggcgcaccggcagtccggcctcgtgcttctgcattgggtcgcgatccaaatcctgggaggctctcctgtcttctctggctatccgaacaggtccgtccgagcattatttgtcagatctctttccgtcttgtcagttgtccacggtctccgagccctcctatttgcatccacgtagattgatgatagattcctctcggaagtgtctcgaaattttctaaactctaaaattagaaagttagtacttgtgaaattctttaaaaaaaaaattactagtaaaactgagctgcgctaaacagatttcgccagatttgaagatctgagatttcgagattatctagtgcagagggacatttcttattttttaccttctcaaatttgaaacactaaacgacttcttttttcagcaggagagactcagattctgatcgcatttttttttcactgatcacatttgtgcttctctctcgtaggccttgtttagttccgaaaagtgaaaactttttggaactgtagcattttcgtttgtttgatcatggactaactaggatcaaaagattcgtcttgtgatttacagctaaactgtgtaattagtttttgttttcgtttatatttaatgtttcatgcatgtgacacaagattcgatgtgacgggaaatcttgaaaactttttggttttcagggtaaactaaacaaggccgtagtcgttctggacaagttttgtcctaacgagcagaagcgtactgacgaaacttgtcttcactggcccattggatcttgtggcagctcatgcaaaaaatatttcgatagttgcctatcacacgcatgtgtttactgtccacagacgctgattctaaagaatcttttcaccatcaattggccgcgacattttggcgatttctctgcgagactcactgatgacacattacttttgcatttattaaaaaatccaatctttttgtctctttttacccgaaaaaagctccctccttttttgacccctcttccttcccctgaaacgcacgcagacgcagctgagcctgaattcttatccacccacggcgccaccctccactcccggaacgtaagccaaaagatcagagaaagagacagacagacaagattaaaagagtgacaaggcaaagagagagaaaaaagagtaagaaaccgccctagaatcttttcagtttttccctgtcttcatgtgtctttgttatagagacgctgccttgtcggggcagcagacttgggacgagagagaggcctagagggggaggagttggtgagcgagctcgggatcagcgggtgaccccagaggttgttgcctctggaagttcccaattctttaacccccatcttcttccacatgactttggactcgctgctgttcttttggttggtatatccttggatctctgactccgcctgttccccgaaatttccagttcttgcttcctcttggaggagggctgcagcctgcaccctgcaggggagagagagcaggaggggacggagacatgggtagcttcgctaagctggcgaagagggcggtggagacggacgcgccggtcatggtgaaggtgacgaatttctcctacctcgtcgaaatcgcatcttgactctggagtttcttgggcttccttggtgagttctttctaataagacgtccctttttcagatacaagaactgcttcgaggggccacggatgtcatgtcgcttgcgcaggtatatgaccataattttctcgatttggctgtgcaatggtttcctgctttgtgatgttatttaaggttaatggaacaagcacgaaacggaaagtcaaacgaaagcagaacggattaatgggagtcgatcatcttatagcaattcgctcccaaatggtttctgtcaattgttcaagttgaaatgtactactgtagttcggtacagttcatttgctacgtttcgtgtaacaaatcatactgtttttacttgctattcctgaatagttcttatgttgccgaagtgggacattcgtatgtaatctaactagtgacatgcgtgtcttttagtgtcctattagcatgacccgaccatatgatatttatttctgagaaattcgtttctgttcttcactttttgtaaatcctgaatgcagggagttgtttactggcaacctcctgagtcagctctggataagatcgaaaaaatcgtcagggaaccaacagtcagtaagtatggttctgatgatggacttcctgagcttcgagaagcacttctcgaaaaggtaacttctttttatcatagaatatataatatattttgcccatacactgactaaagcaggccatgctcttgagtattaccacattgccatgcctttgtcattgggaactagggatcctgcttctaaaccactacaccgtaaccaaattttaatagagatatttgatcttaactcacagtcgcatcagcaatctcaagtgaccgatgcaatggttcagaaattactttgtcaaaacaaaggttccaaaaaatctttttgttcccagagttacattcctgggaagcatctaattttctgaatattggcagagtgtgtcataaaactctctattcatcaactgagaaatgggaacaatattcattattgtgagtttgtgactcctctggaaattcagaagagaaattcatggtggtgctaagagttgcaaagtctctctgccatgttgtttgtctaacagttgagatgaaatcctgagatgatttaggtgctctaaccgttagcctaaatgctccacatcgcacagttatctttagaatttgttccaccgttttgtcacctactgggctaagatttttattgctgtttggaccaccaatgtgttaaaattagtactgcattgcaagcatcttgtatatctttcctgatgtctactgtatttttggcagctacgcagagagaataagcttaccaagtcatcagtcatggtcactgctggtgcaaatcaggtaaaaacttactttcctattttgccttgtaaagttttcaaactgggccttacgtttcccaatttacttatgtttattacttctttaggggcacactcttcctttcctttttcactgctagaccttgaacttgtcttacaatctggtcttttgttacattgattttttcaggcttttgtgaacttggtcctcactctttgtgatgctggtgattctgttgtcatgtttgcaccatattatttcaatgcctacatgtcattccagatgacaggtgttactgacatattaattggtggttgcgatcccaagacacttcatcctgatgttggtaagatgattctcttcttctttctgtttcattggagaaattggtgagatgtcttatatcaatagtctgttgtaagaaatttgttgtacttagtgctggttcaggagcatctgcttcattggcacatgttcaatttccgttataagtctacattatcttttagctgatgagaagatatgcttttgtaaaatacaacgcatctagtataagaaattatgcattggacgtagttgataatatttcattttggaagcatacattacaattatgtctaaaactgcagattggttggagaaggttctgaaagaaaatgaccctatccctaaacttgttactgttgtgaatccggggaacccctctggagcttttgttcccaggcctatgcttgaggtcatctctctccactaacacttgtgtttggaatcactatccaatatttcctaatattgcattgtcattccagagaatttcagatctgtgcaaaaatgctggtgcatggcttgtggttgacaatacctatgagtaagtcaatactgtaaagtgatcattttcacaagcaattatataatcttttgtggtatctgttagagagaacttagaagagccggggaattatggttgtagtcaaagggacagatcatataagccattagagctcttctgcctcctcttgaatctgtgtccagatgaaagacactctgatggcatataggatttctagtatctttgcagtttccacaaggaagggaaaatcagaatattgtgcccataggagattcagaaagtagtcgtcctgcacagatatgttgtgtgagaatatgtcgaatacagttatcctgtgtttgtttttttggtttaggataagttctcttattcaaattcattccagctgccaagcctttataaatacgttttaggctctaaacttggtgatgttaaaaataagccctccaatcacacttactgctttatactttatacaggccagggtttatgtttgcctgaaagagtcaggtgttcaggtccatgttgaggaactaatagtaattaataaaggaccttttccctctaagcttaaagaaacatacaagtttttgtcgtggagctgcgattagccaaccatatttgaagtggtagcataaagaatggagtccaaacatgtcgagtggaacacagaaagtggcttattccttttactccctgcaaacgaattctgctccttattggaatttagttatggtctttagcatttgagctttgtaagatttgatgcttaggtcagttttcctgtgtctttttgcagatactttatgtatgatggaatggagcactattgcttagaagatactcatattgtcaacctcttctcattctcaaaggcttatggaatgatggggtggcgtgtaggatacgtgagtgcatcatactcttcgtttatcattttattatgctttactccactttttggctgctgctgctgttaattgtcactgtaacagtaagtcatacatggactgtctgctgcagattgcatttccaaatgaagctgatggcttccacgatcagctcctcaaagtgcaagacaacatacctatctgtgcctccatcatcgggcagcgcctggcgctctactcactggaggctggccccgagtggatcaaagaaagggtgaaagacttggtgaaaaaccgagcactgctcgtggaggcgctgtccccgctcggtgaggacaatgtgaagggcggggagggcgccatctacctctgggccaaactgccggacaactgctccaacgattttgaagttgtcaggtggcttgcaaacaagcacggtgtcgctgtgatccctggcagcgccagtggaggccccggatacatccgcgtctccttcggagggctcaaagaagaagacaccaggctcgctgctgagaggctaaggcgcggcttgcaggagctggtgactgatggaatggtacagtaa

SEQ ID NO: 3 - protein, 515 aa, Sorghum bicolor
MVHGASATTQFMRAATQQQQQGKDRVPVGAGRQKPPRPRRGIGSSRTAACTTAERQAARPEPDASCPGAPAVRPRASALGRDPNPGRLSCLLWLSEQFLLPLGGGLQPAPCRGERAGGDGDMGSFAKLAKRAVETDAPVMVKIQELLRGATDVMSLAQGVVYWQPPESALDKIEKIVREPTVSKYGSDDGLPELREALLEKLRRENKLTKSSVMVTAGANQAFVNLVLTLCDAGDSVVMFAPYYFNAYMSFQMTGVTDILIGGCDPKTLHPDVDWLEKVLKENDPIPKLVTVVNPGNPSGAFVPRPMLERISDLCKNAGAWLVVDNTYEYFMYDGMEHYCLEDTHIVNLFSFSKAYGMMGWRVGYIAFPNEADGFHDQLLKVQDNIPICASIIGQRLALYSLEAGPEWIKERVKDLVKNRALLVEALSPLGEDNVKGGEGAIYLSAKLPDNCSNDFEVVRWLANKHGVAVIPGSASGGPGYIRVSFGGLKEEDTRLAAERLRRGLQELVTDGMVQ

SEQ ID NO: 4 - protein, 515 aa, Sorghum bicolor
MVHGASATTQFMRAATQQQQQGKDRVPVGAGRQKPPRPRRGIGSSRTAACTTAERQAARPEPDASCPGAPAVRPRASALGRDPNPGRLSCLLWLSEQFLLPLGGGLQPAPCRGERAGGDGDMGSFAKLAKRAVETDAPVMVKIQELLRGATDVMSLAQGVVYWQPPESALDKIEKIVREPTVSKYGSDDGLPELREALLEKLRRENKLTKSSVMVTAGANQAFVNLVLTLCDAGDSVVMFAPYYFNAYMSFQMTGVTDILIGGCDPKTLHPDVDWLEKVLKENDPIPKLVTVVNPGNPSGAFVPRPMLERISDLCKNAGAWLVVDNTYEYFMYDGMEHYCLEDTHIVNLFSFSKAYGMMGWRVGYIAFPNEADGFHDQLLKVQDNIPICASIIGQRLALYSLEAGPEWIKERVKDLVKNRALLVEALSPLGEDNVKGGEGAIYLWAKLPDNCSNDFEVVRWLANKHGVAVIPGSASGGPGYIRVSFGGLKEEDTRLAAERLRRGLQELVTDGMVQ

SEQ ID NO: 5 - DNA, 42 bp, synthetic construct
  source 1..42: mol_type = other DNA
gaaggtgaccaagttcatgctggagggcgccatctacctctc

SEQ ID NO: 6 - DNA, 41 bp, synthetic construct
  source 1..41: mol_type = other DNA
gaaggtcggagtcaacggattgagggcgccatctacctctg

SEQ ID NO: 7 - DNA, 22 bp, synthetic construct
  source 1..22: mol_type = other DNA
gttggagcagttgtccggcagt

SEQ ID NO: 8 - DNA, 101 bp, Sorghum bicolor
  source 1..101: mol_type = genomic DNA
cccgctcggtgaggacaatgtgaagggcggggagggcgccatctacctctcggccaaactgccggacaactgctccaacgattttgaagttgtcaggtggc

SEQ ID NO: 9 - DNA, 101 bp, Sorghum bicolor
  source 1..101: mol_type = genomic DNA
cgaacagtacaatcagccatgcgctgtgtgtcctcaactccactgacttcacaatcaaagaaccccatggacatgcatgtcattccggatctgtgcagcga

SEQ ID NO: 10 - DNA, 101 bp, Sorghum bicolor
  source 1..101: mol_type = genomic DNA
tcacatcgaatcttgtggcacatgcattaagcattaaatatagtcgaaaataaaaacaaattgcacagttgcctgtaaatcgtgagatgaatcttttgagc

SEQ ID NO: 11 - DNA, 2001 bp, Sorghum bicolor
  source 1..2001: mol_type = genomic DNA
cccataggagattcagaaagtagtcgtcctgcacagatatgttgtgtgagaatatgtcgaatacagttatcctgtgtttgtttttttggtttaggataagttctcttattcaaattcattccagctgccaagcctttataaatacgttttaggctctaaacttggtgatgttaaaaataagccctccaatcacacttactgctttatactttatacaggccagggtttatgtttgcctgaaagagtcaggtgttcaggtccatgttgaggaactaatagtaattaataaaggaccttttccctctaagcttaaagaaacatacaagtttttgtcgtggagctgcgattagccaaccatatttgaagtggtagcataaagaatggagtccaaacatgtcgagtggaacacagaaagtggcttattccttttactccctgcaaacgaattctgctccttattggaatttagttatggtctttagcatttgagctttgtaagatttgatgcttaggtcagttttcctgtgtctttttgcagatactttatgtatgatggaatggagcactattgcttagaagatactcatattgtcaacctcttctcattctcaaaggcttatggaatgatggggtggcgtgtaggatacgtgagtgcatcatactcttcgtttatcattttattatgctttactccactttttggctgctgctgctgttaattgtcactgtaacagtaagtcatacatggactgtctgctgcagattgcatttccaaatgaagctgatggcttccacgatcagctcctcaaagtgcaagacaacatacctatctgtgcctccatcatcgggcagcgcctggcgctctactcactggaggctggccccgagtggatcaaagaaagggtgaaagacttggtgaaaaaccgagcactgctcgtggaggcgctgtccccgctcggtgaggacaatgtgaagggcggggagggcgccatctacctctsggccaaactgccggacaactgctccaacgattttgaagttgtcaggtggcttgcaaacaagcacggtgtcgctgtgatccctggcagcgccagtggaggccccggatacatccgcgtctccttcggagggctcaaagaagaagacaccaggctcgctgctgagaggctaaggcgcggcttgcaggagctggtgactgatggaatggtacagtaactggtcccttggtgtaagtaaaaacgtagcattgaagttttacagaactccaattttccaatggtataattctataccgggattaattcgagagaagactattttcaagaatacattgttcggacgagagccggggacagaataaataaaaggtatccggtctgcagagtaacctatgttataaaatataaatagtacatatatcatcagtgagtacttgtttctgtttgacattcagttatctatattactgatggcagcaattgtacacagtcaaaagtataagcaagaattgtgagctgctcttgacgtacagagctgcctgttagtaatttgcttggcttagcttgagaatgaatactaatgtccaaataacgtcagcatctgcctggataattcataattcaagtgtctgcaagtaagatagatctactatatctataaagcagtgttaaaaaggactcctcaagtttgcacatagaaattacgggattaataaaaaatgcagttggattttaagataatctaatggtatagatgtactagaaattaccagaaaatagaaccaatggttttttttaatgacctaatagtctaaatccaaaggaaaatgtatgccaaatataatctgcatttaaactttttccgtacctacggtgctgtaagccaaaaactcttctcctattttaataaacacgtcttcaaggaaaaatataaaagtcatcaagaccaataaagtttatttatagaaaagttacagacatcaaattacggttggaagaatag

SEQ ID NO: 12 - DNA, 1548 bp, Sorghum bicolor
  source 1..1548: mol_type = genomic DNA
atggttcacggcgcctccgcgacaacgcaattcatgcgcgccgcgacgcagcagcagcagcaaggaaaggaccgcgtgcccgtgggcgccggtcgacaaaaacctcctcgcccccggaggggcatcggatccagccgaaccgcagcgtgtacgaccgcggagcggcaagcggcgcgacccgaacccgacgcgagctgcccgggcgcaccggcagtccggcctcgtgcttctgcattgggtcgcgatccaaatcctgggaggctctcctgtcttctctggctatccgaacagttcttgcttcctcttggaggagggctgcagcctgcaccctgcaggggagagagagcaggaggggacggagacatgggtagcttcgctaagctggcgaagagggcggtggagacggacgcgccggtcatggtgaagatacaagaactgcttcgaggggccacggatgtcatgtcgcttgcgcagggagttgtttactggcaacctcctgagtcagctctggataagatcgaaaaaatcgtcagggaaccaacagtcagtaagtatggttctgatgatggacttcctgagcttcgagaagcacttctcgaaaagctacgcagagagaataagcttaccaagtcatcagtcatggtcactgctggtgcaaatcaggcttttgtgaacttggtcctcactctttgtgatgctggtgattctgttgtcatgtttgcaccatattatttcaatgcctacatgtcattccagatgacaggtgttactgacatattaattggtggttgcgatcccaagacacttcatcctgatgttgattggttggagaaggttctgaaagaaaatgaccctatccctaaacttgttactgttgtgaatccggggaacccctctggagcttttgttcccaggcctatgcttgagagaatttcagatctgtgcaaaaatgctggtgcatggcttgtggttgacaatacctatgaatactttatgtatgatggaatggagcactattgcttagaagatactcatattgtcaacctcttctcattctcaaaggcttatggaatgatggggtggcgtgtaggatacattgcatttccaaatgaagctgatggcttccacgatcagctcctcaaagtgcaagacaacatacctatctgtgcctccatcatcgggcagcgcctggcgctctactcactggaggctggccccgagtggatcaaagaaagggtgaaagacttggtgaaaaaccgagcactgctcgtggaggcgctgtccccgctcggtgaggacaatgtgaagggcggggagggcgccatctacctctgggccaaactgccggacaactgctccaacgattttgaagttgtcaggtggcttgcaaacaagcacggtgtcgctgtgatccctggcagcgccagtggaggccccggatacatccgcgtctccttcggagggctcaaagaagaagacaccaggctcgctgctgagaggctaaggcgcggcttgcaggagctggtgactgatggaatggtacagtaa

SEQ ID NO: 13 - DNA, 1548 bp, Sorghum bicolor
  source 1..1548: mol_type = genomic DNA
atggttcacggcgcctccgcgacaacgcaattcatgcgcgccgcgacgcagcagcagcagcaaggaaaggaccgcgtgcccgtgggcgccggtcgacaaaaacctcctcgcccccggaggggcatcggatccagccgaaccgcagcgtgtacgaccgcggagcggcaagcggcgcgacccgaacccgacgcgagctgcccgggcgcaccggcagtccggcctcgtgcttctgcattgggtcgcgatccaaatcctgggaggctctcctgtcttctctggctatccgaacagttcttgcttcctcttggaggagggctgcagcctgcaccctgcaggggagagagagcaggaggggacggagacatgggtagcttcgctaagctggcgaagagggcggtggagacggacgcgccggtcatggtgaagatacaagaactgcttcgaggggccacggatgtcatgtcgcttgcgcagggagttgtttactggcaacctcctgagtcagctctggataagatcgaaaaaatcgtcagggaaccaacagtcagtaagtatggttctgatgatggacttcctgagcttcgagaagcacttctcgaaaagctacgcagagagaataagcttaccaagtcatcagtcatggtcactgctggtgcaaatcaggcttttgtgaacttggtcctcactctttgtgatgctggtgattctgttgtcatgtttgcaccatattatttcaatgcctacatgtcattccagatgacaggtgttactgacatattaattggtggttgcgatcccaagacacttcatcctgatgttgattggttggagaaggttctgaaagaaaatgaccctatccctaaacttgttactgttgtgaatccggggaacccctctggagcttttgttcccaggcctatgcttgagagaatttcagatctgtgcaaaaatgctggtgcatggcttgtggttgacaatacctatgaatactttatgtatgatggaatggagcactattgcttagaagatactcatattgtcaacctcttctcattctcaaaggcttatggaatgatggggtggcgtgtaggatacattgcatttccaaatgaagctgatggcttccacgatcagctcctcaaagtgcaagacaacatacctatctgtgcctccatcatcgggcagcgcctggcgctctactcactggaggctggccccgagtggatcaaagaaagggtgaaagacttggtgaaaaaccgagcactgctcgtggaggcgctgtccccgctcggtgaggacaatgtgaagggcggggagggcgccatctacctctcggccaaactgccggacaactgctccaacgattttgaagttgtcaggtggcttgcaaacaagcacggtgtcgctgtgatccctggcagcgccagtggaggccccggatacatccgcgtctccttcggagggctcaaagaagaagacaccaggctcgctgctgagaggctaaggcgcggcttgcaggagctggtgactgatggaatggtacagtaa

SEQ ID NO: 14 - DNA, 1548 bp, Sorghum bicolor
  source 1..1548: mol_type = genomic DNA
atggttcacggcgcctccgcgacaacgcaattcatgcgcgccgcgacgcagcagcagcagcaaggaaaggaccgcgtgcccgtgggcgccggtcgacaaaaacctcctcgcccccggaggggcatcggatccagccgaaccgcagcgtgtacgaccgcggagcggcaagcggcgcgacccgaacccgacgcgagctgcccgggcgcaccggcagtccggcctcgtgcttctgcattgggtcgcgatccaaatcctgggaggctctcctgtcttctctggctatccgaacagttcttgcttcctcttggaggagggctgcagcctgcaccctgcaggggagagagagcaggaggggacggagacatgggtagcttcgctaagctggcgaagagggcggtggagacggacgcgccggtcatggtgaagatacaagaactgcttcgaggggccacggatgtcatgtcgcttgcgcagggagttgtttactggcaacctcctgagtcagctctggataagatcgaaaaaatcgtcagggaaccaacagtcagtaagtatggttctgatgatggacttcctgagcttcgagaagcacttctcgaaaagctacgcagagagaataagcttaccaagtcatcagtcatggtcactgctggtgcaaatcaggcttttgtgaacttggtcctcactctttgtgatgctggtgattctgttgtcatgtttgcaccatattatttcaatgcctacatgtcattccagatgacaggtgttactgacatattaattggtggttgcgatcccaagacacttcatcctgatgttgattggttggagaaggttctgaaagaaaatgaccctatccctaaacttgttactgttgtgaatccggggaacccctctggagcttttgttcccaggcctatgcttgagagaatttcagatctgtgcaaaaatgctggtgcatggcttgtggttgacaatacctatgaatactttatgtatgatggaatggagcactattgcttagaagatactcatattgtcaacctcttctcattctcaaaggcttatggaatgatggggtggcgtgtaggatacattgcatttccaaatgaagctgatggcttccacgatcagctcctcaaagtgcaagacaacatacctatctgtgcctccatcatcgggcagcgcctggcgctctactcactggaggctggccccgagtggatcaaagaaagggtgaaagacttggtgaaaaaccgagcactgctcgtggaggcgctgtccccgctcggtgaggacaatgtgaagggcggggagggcgccatctacctctctgccaaactgccggacaactgctccaacgattttgaagttgtcaggtggcttgcaaacaagcacggtgtcgctgtgatccctggcagcgccagtggaggccccggatacatccgcgtctccttcggagggctcaaagaagaagacaccaggctcgctgctgagaggctaaggcgcggcttgcaggagctggtgactgatggaatggtacagtaa

SEQ ID NO: 15 - DNA, 1548 bp, Sorghum bicolor
  source 1..1548: mol_type = genomic DNA
atggttcacggcgcctccgcgacaacgcaattcatgcgcgccgcgacgcagcagcagcagcaaggaaaggaccgcgtgcccgtgggcgccggtcgacaaaaacctcctcgcccccggaggggcatcggatccagccgaaccgcagcgtgtacgaccgcggagcggcaagcggcgcgacccgaacccgacgcgagctgcccgggcgcaccggcagtccggcctcgtgcttctgcattgggtcgcgatccaaatcctgggaggctctcctgtcttctctggctatccgaacagttcttgcttcctcttggaggagggctgcagcctgcaccctgcaggggagagagagcaggaggggacggagacatgggtagcttcgctaagctggcgaagagggcggtggagacggacgcgccggtcatggtgaagatacaagaactgcttcgaggggccacggatgtcatgtcgcttgcgcagggagttgtttactggcaacctcctgagtcagctctggataagatcgaaaaaatcgtcagggaaccaacagtcagtaagtatggttctgatgatggacttcctgagcttcgagaagcacttctcgaaaagctacgcagagagaataagcttaccaagtcatcagtcatggtcactgctggtgcaaatcaggcttttgtgaacttggtcctcactctttgtgatgctggtgattctgttgtcatgtttgcaccatattatttcaatgcctacatgtcattccagatgacaggtgttactgacatattaattggtggttgcgatcccaagacacttcatcctgatgttgattggttggagaaggttctgaaagaaaatgaccctatccctaaacttgttactgttgtgaatccggggaacccctctggagcttttgttcccaggcctatgcttgagagaatttcagatctgtgcaaaaatgctggtgcatggcttgtggttgacaatacctatgaatactttatgtatgatggaatggagcactattgcttagaagatactcatattgtcaacctcttctcattctcaaaggcttatggaatgatggggtggcgtgtaggatacattgcatttccaaatgaagctgatggcttccacgatcagctcctcaaagtgcaagacaacatacctatctgtgcctccatcatcgggcagcgcctggcgctctactcactggaggctggccccgagtggatcaaagaaagggtgaaagacttggtgaaaaaccgagcactgctcgtggaggcgctgtccccgctcggtgaggacaatgtgaagggcggggagggcgccatctacctctccgccaaactgccggacaactgctccaacgattttgaagttgtcaggtggcttgcaaacaagcacggtgtcgctgtgatccctggcagcgccagtggaggccccggatacatccgcgtctccttcggagggctcaaagaagaagacaccaggctcgctgctgagaggctaaggcgcggcttgcaggagctggtgactgatggaatggtacagtaa

SEQ ID NO: 16 - DNA, 1548 bp, Sorghum bicolor
  source 1..1548: mol_type = genomic DNA
atggttcacggcgcctccgcgacaacgcaattcatgcgcgccgcgacgcagcagcagcagcaaggaaaggaccgcgtgcccgtgggcgccggtcgacaaaaacctcctcgcccccggaggggcatcggatccagccgaaccgcagcgtgtacgaccgcggagcggcaagcggcgcgacccgaacccgacgcgagctgcccgggcgcaccggcagtccggcctcgtgcttctgcattgggtcgcgatccaaatcctgggaggctctcctgtcttctctggctatccgaacagttcttgcttcctcttggaggagggctgcagcctgcaccctgcaggggagagagagcaggaggggacggagacatgggtagcttcgctaagctggcgaagagggcggtggagacggacgcgccggtcatggtgaagatacaagaactgcttcgaggggccacggatgtcatgtcgcttgcgcagggagttgtttactggcaacctcctgagtcagctctggataagatcgaaaaaatcgtcagggaaccaacagtcagtaagtatggttctgatgatggacttcctgagcttcgagaagcacttctcgaaaagctacgcagagagaataagcttaccaagtcatcagtcatggtcactgctggtgcaaatcaggcttttgtgaacttggtcctcactctttgtgatgctggtgattctgttgtcatgtttgcaccatattatttcaatgcctacatgtcattccagatgacaggtgttactgacatattaattggtggttgcgatcccaagacacttcatcctgatgttgattggttggagaaggttctgaaagaaaatgaccctatccctaaacttgttactgttgtgaatccggggaacccctctggagcttttgttcccaggcctatgcttgagagaatttcagatctgtgcaaaaatgctggtgcatggcttgtggttgacaatacctatgaatactttatgtatgatggaatggagcactattgcttagaagatactcatattgtcaacctcttctcattctcaaaggcttatggaatgatggggtggcgtgtaggatacattgcatttccaaatgaagctgatggcttccacgatcagctcctcaaagtgcaagacaacatacctatctgtgcctccatcatcgggcagcgcctggcgctctactcactggaggctggccccgagtggatcaaagaaagggtgaaagacttggtgaaaaaccgagcactgctcgtggaggcgctgtccccgctcggtgaggacaatgtgaagggcggggagggcgccatctacctctcagccaaactgccggacaactgctccaacgattttgaagttgtcaggtggcttgcaaacaagcacggtgtcgctgtgatccctggcagcgccagtggaggccccggatacatccgcgtctccttcggagggctcaaagaagaagacaccaggctcgctgctgagaggctaaggcgcggcttgcaggagctggtgactgatggaatggtacagtaa

SEQ ID NO: 17 - DNA, 1548 bp, Sorghum bicolor
  source 1..1548: mol_type = genomic DNA
atggttcacggcgcctccgcgacaacgcaattcatgcgcgccgcgacgcagcagcagcagcaaggaaaggaccgcgtgcccgtgggcgccggtcgacaaaaacctcctcgcccccggaggggcatcggatccagccgaaccgcagcgtgtacgaccgcggagcggcaagcggcgcgacccgaacccgacgcgagctgcccgggcgcaccggcagtccggcctcgtgcttctgcattgggtcgcgatccaaatcctgggaggctctcctgtcttctctggctatccgaacagttcttgcttcctcttggaggagggctgcagcctgcaccctgcaggggagagagagcaggaggggacggagacatgggtagcttcgctaagctggcgaagagggcggtggagacggacgcgccggtcatggtgaagatacaagaactgcttcgaggggccacggatgtcatgtcgcttgcgcagggagttgtttactggcaacctcctgagtcagctctggataagatcgaaaaaatcgtcagggaaccaacagtcagtaagtatggttctgatgatggacttcctgagcttcgagaagcacttctcgaaaagctacgcagagagaataagcttaccaagtcatcagtcatggtcactgctggtgcaaatcaggcttttgtgaacttggtcctcactctttgtgatgctggtgattctgttgtcatgtttgcaccatattatttcaatgcctacatgtcattccagatgacaggtgttactgacatattaattggtggttgcgatcccaagacacttcatcctgatgttgattggttggagaaggttctgaaagaaaatgaccctatccctaaacttgttactgttgtgaatccggggaacccctctggagcttttgttcccaggcctatgcttgagagaatttcagatctgtgcaaaaatgctggtgcatggcttgtggttgacaatacctatgaatactttatgtatgatggaatggagcactattgcttagaagatactcatattgtcaacctcttctcattctcaaaggcttatggaatgatggggtggcgtgtaggatacattgcatttccaaatgaagctgatggcttccacgatcagctcctcaaagtgcaagacaacatacctatctgtgcctccatcatcgggcagcgcctggcgctctactcactggaggctggccccgagtggatcaaagaaagggtgaaagacttggtgaaaaaccgagcactgctcgtggaggcgctgtccccgctcggtgaggacaatgtgaagggcggggagggcgccatctacctcagtgccaaactgccggacaactgctccaacgattttgaagttgtcaggtggcttgcaaacaagcacggtgtcgctgtgatccctggcagcgccagtggaggccccggatacatccgcgtctccttcggagggctcaaagaagaagacaccaggctcgctgctgagaggctaaggcgcggcttgcaggagctggtgactgatggaatggtacagtaa

SEQ ID NO: 18 - DNA, 1548 bp, Sorghum bicolor
  source 1..1548: mol_type = genomic DNA
atggttcacggcgcctccgcgacaacgcaattcatgcgcgccgcgacgcagcagcagcagcaaggaaaggaccgcgtgcccgtgggcgccggtcgacaaaaacctcctcgcccccggaggggcatcggatccagccgaaccgcagcgtgtacgaccgcggagcggcaagcggcgcgacccgaacccgacgcgagctgcccgggcgcaccggcagtccggcctcgtgcttctgcattgggtcgcgatccaaatcctgggaggctctcctgtcttctctggctatccgaacagttcttgcttcctcttggaggagggctgcagcctgcaccctgcaggggagagagagcaggaggggacggagacatgggtagcttcgctaagctggcgaagagggcggtggagacggacgcgccggtcatggtgaagatacaagaactgcttcgaggggccacggatgtcatgtcgcttgcgcagggagttgtttactggcaacctcctgagtcagctctggataagatcgaaaaaatcgtcagggaaccaacagtcagtaagtatggttctgatgatggacttcctgagcttcgagaagcacttctcgaaaagctacgcagagagaataagcttaccaagtcatcagtcatggtcactgctggtgcaaatcaggcttttgtgaacttggtcctcactctttgtgatgctggtgattctgttgtcatgtttgcaccatattatttcaatgcctacatgtcattccagatgacaggtgttactgacatattaattggtggttgcgatcccaagacacttcatcctgatgttgattggttggagaaggttctgaaagaaaatgaccctatccctaaacttgttactgttgtgaatccggggaacccctctggagcttttgttcccaggcctatgcttgagagaatttcagatctgtgcaaaaatgctggtgcatggcttgtggttgacaatacctatgaatactttatgtatgatggaatggagcactattgcttagaagatactcatattgtcaacctcttctcattctcaaaggcttatggaatgatggggtggcgtgtaggatacattgcatttccaaatgaagctgatggcttccacgatcagctcctcaaagtgcaagacaacatacctatctgtgcctccatcatcgggcagcgcctggcgctctactcactggaggctggccccgagtggatcaaagaaagggtgaaagacttggtgaaaaaccgagcactgctcgtggaggcgctgtccccgctcggtgaggacaatgtgaagggcggggagggcgccatctacctcagcgccaaactgccggacaactgctccaacgattttgaagttgtcaggtggcttgcaaacaagcacggtgtcgctgtgatccctggcagcgccagtggaggccccggatacatccgcgtctccttcggagggctcaaagaagaagacaccaggctcgctgctgagaggctaaggcgcggcttgcaggagctggtgactgatggaatggtacagtaa

SEQ ID NO: 19 - DNA, 101 bp, Sorghum bicolor
  source 1..101: mol_type = genomic DNA
cccgctcggtgaggacaatgtgaagggcggggagggcgccatctacctctgggccaaactgccggacaactgctccaacgattttgaagttgtcaggtggc

SEQ ID NO: 20 - DNA, 101 bp, Sorghum bicolor
  source 1..101: mol_type = genomic DNA
cgaacagtacaatcagccatgcgctgtgtgtcctcaactccactgacttcgcaatcaaagaaccccatggacatgcatgtcattccggatctgtgcagcga

SEQ ID NO: 21 - DNA, 101 bp, Sorghum bicolor
  source 1..101: mol_type = genomic DNA
tcacatcgaatcttgtggcacatgcattaagcattaaatatagtcgaaaacaaaaacaaattgcacagttgcctgtaaatcgtgagatgaatcttttgagc